(19) United States
(12) Patent Application Publication
(10) Pub. No.: US 2023/0337672 A1
(43) Pub. Date: Oct. 26, 2023

(54) METHOD OF CONTROLLING SOYBEAN RUST FUNGUS HAVING RESISTANCE TO QoI FUNGICIDE

(71) Applicant: SUMITOMO CHEMICAL COMPANY, LIMITED, Chuo-ku (JP)

(72) Inventors: Hidekatsu TOBITA, Takarazuka-shi (JP); Takaaki NAKANO, Kasai-shi (JP); Yoshihiko NOKURA, Takarazuka-shi (JP)

(73) Assignee: SUMITOMO CHEMICAL COMPANY, LIMITED, Chuo-ku (JP)

(21) Appl. No.: 18/002,617

(22) PCT Filed: Jun. 29, 2021
(86) PCT No.: PCT/JP2021/024490

§ 371 Date: Dec. 20, 2022

(30) Foreign Application Priority Data
Jun. 30, 2020 (JP) 2020-112466

(51) Int. Cl. A01N 43/76; A01N 43/653; A01N 43/78; A01P 3/00; A01N 37/18

(52) U.S. Cl. CPC A01N 43/78; A01N 37/18; A01N 43/653; A01N 43/76; A01P 3/00

(57) ABSTRACT

A method for controlling a soybean rust fungus having an amino acid substitution of F129L in a mitochondrial cytochrome b protein. A compound of formula (I) can be used for controlling a soybean rust fungus having an amino acid substitution of F129L in a mitochondrial cytochrome b protein,

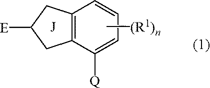

wherein: R¹ represents a C1-C4 alkyl group or the like; n represents 0, 1, or 2; Q represents the group of Q1 or the like;

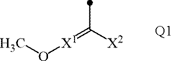

represents the binding site to the rest of molecule; X¹ represents —C(H)═ or the like; X² represents -C(O)OCH₃ or the like; J represents the group represented by J1 or the like;

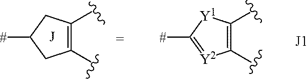

# represents the binding position to E; Y¹ represents an oxygen atom or the like; Y² represents ═C(R⁶)— or the like; R⁶ represents a C1-C4 alkyl group or the like; and E represents a C1-C6 chain hydrocarbon group or the like.

### TECHNICAL FIELD

This application claims the priority to and the benefit of Japanese Patent Application No. 2020-112466 filed on Jun. 30, 2020, the entire contents of which are incorporated herein by reference.

The present invention relates to a method for controlling a soybean rust fungus having an amino acid substitution of F129L in a mitochondrial cytochrome b protein.

### BACKGROUND ART

The spread of phytopathogenic fungi that shows acquired character being resistant to agricultural fungicides becomes a major problem. Under such circumstances, FRAC (Fungicide Resistance Action Committee) has been established as an organization that provides guidelines for acquiring a resistance to existing agricultural fungicides, and suppressing and delaying the spread of the fungi having the resistance acquired. A variety of information on phytopathogenic fungi that shows a resistance to agricultural fungicides is available on the FRAC-provided website (http://www.frac.info/).

It has been known that in the case of a phytopathogenic fungi, the main cause of acquiring a resistance is that a mutation of the phytopathogenic fungal gene encoding the target enzyme of the fungicide causes a partial substitution of amino acids in the target enzyme of the fungicides, which results in reducing the affinity between the fungicides and the target enzyme.

QoI fungicides are named as aliases a strobilurin fungicide, or a methoxyacrylate fungicide because of its characteristic structure. QoI fungicides are one group of agricultural fungicides that have been widely used to control phytopathogenic fungi including soybean rust fungus. QoI fungicides usually bind to the ubihydroquinone oxidation centers of cytochrome bc1 complex (electron transfer complex III) in mitochondria, and suppress a respiration of the phytopathogenic fungi, which results in killing the phytopathogenic fungi or stopping the growth of the same. The above-mentioned oxidation center is located outside the mitochondrial inner membrane (see NON-PATENT DOCUMENT 1).

It has been revealed by model studies in the laboratory before QoI fungicides were actually used extensively as agricultural fungicides that phytopathogenic fungi are subjected to a selection pressure by QoI fungicide, which results in easily generating the fungi having a resistance to a QoI fungicide that has acquired a gene mutation that causes a specific single amino acid substitution such as G143A in the cytochrome b gene of the target enzyme cytochrome bc1 complex (see NON-PATENT DOCUMENTs 2 to 4).

On the other hand, soybean rust fungus (scientific name: Phakopsora pachyrhizi) is a phytopathogenic fungus that causes damages to soybeans. Since QoI fungicides have been widely used for controlling soybean rust fungi, an emergence of soybean rust fungi showing a resistance to the QoI fungicides has been reported (see NON-PATENT DOCUMENT 5).

Regarding soybean rust fungi, a strain which has acquired a gene mutation causing a single amino acid substitution of F129L in the same cytochrome b gene has become a problem as a resistant fungus against QoI fungicides. The efficacy of the QoI fungicides conventionally used against soybean rust fungi, that is, pyribencarb, azoxystrobin, dimoxystrobin, metominostrobin, fluoxastrobin, kresoxim-methyl, and the others, has been reduced to the level of practical problems against said resistant fungi (see NON-PATENT DOCUMENT 6).

### CITATION LIST

### Non-patent Document

NON-PATENT DOCUMENT 1: Sauter, “Modern Crop Protection Compounds”, Vol.2, Wiley-VCH Verlag, 2007, p.457-495: Chapter 13.2, Strobilurins and other complex III inhibitors
NON-PATENT DOCUMENT 2: “Journal of Biological Chemistry”, 1989, Vol.264, No.24, p.14543-14548
NON-PATENT DOCUMENT 3: “Genetics”, 1991, Vol.127, p.335-343
NON-PATENT DOCUMENT 4: “Current Genetics”, 2000, Vol.38, p.148-155
NON-PATENT DOCUMENT 5: “Pest Management Science”, 2014, Vol.70, No.3, p.378-388
NON-PATENT DOCUMENT 6: “Pesq. agropec. bras.” (Brasilia), 2016, Vol.51, No.5, p.407-421

### SUMMARY OF THE INVENTION

### Problems to Be Solved by the Invention

On the basis of these facts, an object of the present invention is to provide a method for controlling a soybean rust fungus having an amino acid substitution of F129L in a mitochondrial cytochrome b protein.

### Means to Solve Problems

The present invention provides the followings.

A method for controlling a soybean rust fungus having an amino acid substitution of F129L in a mitochondrial cytochrome b protein, which comprises applying an effective amount of a compound represented by formula (I)

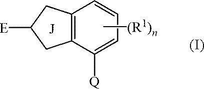

[wherein:
R¹ represents a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, or a hydroxy group;
n represents 0, 1, or 2;
when n represents 2, two R¹ may be identical to or different from each other;
Q represents a group represented by Q1 or a group
represented by Q2;

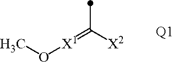

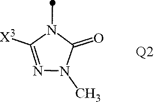

represents the binding site to the rest of molecule;
X¹ represents —C(H)═ or —N═;
X² represents —C(O)OCH₃, —C(O)NHCH₃, or a 5,6-dihydro-1,4,2-dioxazin-3-yl group;
X³ represents a C1-C3 chain hydrocarbon group, a cyclopropyl group, a C1-C3 alkoxy group {wherein said C1-C3 chain hydrocarbon group, said cyclopropyl group, and said C1-C3 alkoxy group are optionally substituted with one or more halogen atom(s)}, or a halogen atom;
J represents a group represented by J1 or a group represented by J2;

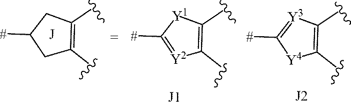

# represents the binding position to E;
Y¹ represents an oxygen atom, a sulfur atom, —N(R²)—, *—C(R³)═C(R⁴)—, or *—N═C(R⁵)—,
* represents the binding position to the carbon atom bound to E;
Y² represents ═C(R⁶)— or ═N—;
Y³ represents ═C(R⁷)— or ═N—;
Y⁴ represents an oxygen atom, a sulfur atom, or —N(R⁸)—,
R² and R⁸ are identical to or different from each other, and each represent a C1-C3 chain hydrocarbon group, a cyclopropyl group {wherein said C1-C3 chain hydrocarbon group and said cyclopropyl group are optionally substituted with one or more halogen atom(s)}, or a hydrogen atom;
R³, R⁴, R⁵, R⁶, and R⁷ are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;
E represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group D}, R⁹—L¹—CH₂—, R¹⁰R¹¹C═N—O—CH₂—, R¹²O—N═C(R¹³)—C(R¹⁴)═N—O—CH₂—, R¹⁵C(O)—C(R¹⁶)═N—O—CH₂—, R¹⁷R¹⁸N—C(S)—O—CH₂—, R¹⁹N═C(R²⁰)—S—CH₂—, R²¹N═C(SR²²)—S—CH₂—, R²³O—N═C(R²⁴)—S—CH₂—, R²⁵O—N═C(SR²⁶)—S—CH₂—, R²⁷O—N═C(R²⁸)—, R²⁹R³⁰C═N—N═C(R³¹)—, R³²R³³N—N═C(R³⁴)—, R³⁵—N═C(R³⁶)—, R³⁷SC(R³⁸)═N—, R³⁹SC(SR⁴⁰)═N—, R⁴¹L²-, R⁴³C(O)O—, R⁴⁴OC(O)O—, R⁴⁵R⁴⁶NC(O)O—, R⁴⁷R⁴⁸NC(S)O—, R⁴⁹S(O)₂O—, R⁵⁰R⁵¹NS(O)₂O-, a cyano group, a nitro group, a hydroxy group, or a halogen atom;
L¹ and L² are identical to or different from each other, and each represent an oxygen atom or a sulfur atom;
R⁹ represents a C6-C10 aryl group or a 5-10 membered aromatic heterocyclic group {wherein said C6-C10 aryl group and said 5-10 membered aromatic heterocyclic group are optionally substituted with one or more substituent(s) selected from Group D};
R¹², R¹⁵, R¹⁷, R¹⁹, R²¹, R²³, R²⁵, R²⁹, R³², R³⁷, R³⁹, R⁴³, R⁴⁴, R⁴⁵, R⁴⁷, R⁴⁹, and R⁵⁰ are identical to or different from each other, and each represent a C1-C6 chain hydrocarbon group optionally substituted with one or more substituent(s) selected from Group F, a C3-C10 alicyclic hydrocarbon group optionally substituted with one or more substituent(s) selected from Group B, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C};
R¹⁰, R²⁷, R³⁵, and R⁴¹ are identical to or different from each other, and each represent a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D};
R¹¹, R¹³, R¹⁴, R¹⁶, R¹⁸, R²⁰, R²², R²⁴, R²⁶, R²⁸, R³⁰, R³¹, R³³, R³⁴, R³⁶, R³⁸, R⁴⁰, R⁴⁶, R⁴⁸, and R⁵¹ are identical to or different from each other, and each represent a C1-C3 chain hydrocarbon group optionally substituted with one or more halogen atom(s), a cyclopropyl group, or a hydrogen atom;
R¹⁰ and R¹¹ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}; and
R²⁹ and R³⁰ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B};
Group A: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C1-C4 alkoxy group, a C1-C4 alkylthio group {wherein said C1-C4 alkoxy group and said C1-C4 alkylthio group are optionally substituted with one or more substituent(s) selected from Group F}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D};
Group B: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;
Group C: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F}, a C3-C6 cycloalkyl group {wherein said C3-C6 cycloalkyl group is optionally substituted with one or more substituent(s) selected from Group B}, a cyano group, a nitro group, a halogen atom, and a hydroxy group;
Group D: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C}, a halogen atom, a cyano group, a nitro group, a hydroxy group, and an amino group;
Group F: a group consisting of a C3-C4 cycloalkyl group, a halogen atom, and a C1-C3 alkoxy group] or an N-oxide or an agriculturally acceptable salt thereof (hereinafter the compound represented by formula (I), or an N-oxide or an agriculturally acceptable salt thereof is referred to as “Present compound”) to a soybean or soil for cultivating a soybean.

The method according to [1], wherein
Q represents the group represented by Q1; J represents the group represented by J1; and
n represents 0
in the compound represented by formula (I), or an N-oxide or an agriculturally acceptable salt thereof.

The method according to [1] or [2], wherein

E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a phenyl group, a 5-6 membered aromatic heterocyclic group {wherein said phenyl group and said 5-6 membered aromatic heterocyclic group are optionally substituted with one or more substituent(s) selected from Group D}, R⁹—L¹—CH₂—, R⁴¹L²—, or a halogen atom in the compound represented by formula (I), or an N-oxide or an agriculturally acceptable salt thereof.

Use of the compound represented by formula (I), or an N-oxide or an agriculturally acceptable salt thereof as defined in any one of [1] to [3] for controlling a soybean rust fungus having an amino acid substitution of F129L in a mitochondrial cytochrome b protein.

### EFFECT OF INVENTION

According to the present invention, a soybean rust fungus having an amino acid substitution of F129L in a mitochondrial cytochrome b protein can be controlled.

### MODE FOR CARRYING OUT THE INVENTION

The substituents in the present invention are explained as follows.

The term of “halogen atom” represents a fluorine atom, a chlorine atom, a bromine atom, or an iodine atom.

When a substituent is substituted with two or more halogen atoms, these halogen atoms may be identical to or different from each other.

When a substituent is substituted with two or more groups or atoms selected from a specific group (for example, a group consisting of a C1-C3 alkyl group and a halogen atom), these groups or atoms may be identical to or different from each other.

The expression of “optionally substituted with one or more substituent(s) selected from Group X” (wherein X represents any one of A, B, C, D, and F) as described herein means that when two or more substituents are selected from Group X, these substituents may be identical to or different from each other.

The expression of “CX-CY” as described herein means that the number of carbon atom is X to Y. For example, the expression of “C1-C6” means that the number of carbon atom is 1 to 6.

The term of “chain hydrocarbon group” represents an alkyl group, an alkenyl group, or an alkynyl group.

Examples of the term of “alkyl group” include a methyl group, an ethyl group, a propyl group, an isopropyl group, a 1,1-dimethylpropyl group, a 1,2-dimethylpropyl group, a 1-ethylpropyl group, a butyl group, a sec-butyl group, a tert-butyl group, a pentyl group, and a hexyl group.

Examples of the term of “alkenyl group” include a vinyl group, a 1-propenyl group, a 2-propenyl group, a 1-methyl-1-propenyl group, a 1-methyl-2-propenyl group, a 1,2-dimethyl-1-propenyl group, a 1-ethyl-2-propenyl group, a 3-butenyl group, a 4-pentenyl group, and a 5-hexenyl group.

Examples of the term of “alkynyl group” include an ethynyl group, a 1-propynyl group, a 2-propynyl group, a 1-methyl-2-propynyl group, a 1,1-dimethyl-2-propynyl group, a 1-ethyl-2-propynyl group, a 2-butynyl group, a 4-pentynyl group, and a 5-hexynyl group.

Examples of the term of “alkoxy group” include a methoxy group, an ethoxy group, a propoxy group, an isopropoxy group, a butoxy group, a tert-butoxy group, a pentyloxy group, and a hexyloxy group.

Examples of the term of “alkylthio group” include a methylthio group, an ethylthio group, a propylthio group, an isopropylthio group, a butylthio group, a pentylthio group, and a hexylthio group.

Examples of the term of “alkylamino group” include a methylamino group, an ethylamino group, an isopropylamino group, and a hexylamino group.

Examples of the term of “dialkylamino group” include a dimethylamino group, a methylethylamino group, a diisopropylamino group, a methylheptylamino group, and a dibutylamino group.

Examples of the term of “(C1-C6 alkyl)carbonyl group” include an acetyl group, a propanoyl group, a 2-methylpropanoyl group, and a heptanoyl group.

Examples of the term of “C2-C4 alkylcarbonyl group” include an acetyl group, a propanoyl group, and a 2-methylpropanoyl group.

Examples of the term of “(C1-C6 alkoxy)carbonyl group” include a methoxycarbonyl group, an isopropoxycarbonyl group, and a hexyloxycarbonyl group.

Examples of the term of “C2-C4 alkoxycarbonyl group” include a methoxycarbonyl group and an isopropoxycarbonyl group.

Examples of the term of “(C1-C6 alkylamino)carbonyl group” include a methylaminocarbonyl group, an isopropylaminocarbonyl group, and a hexylaminocarbonyl group.

Examples of the term of “(C1-C3 alkylamino)carbonyl group” include a methylaminocarbonyl group and an isopropylaminocarbonyl group.

Examples of the term of “(C2-C8 dialkylamino)carbonyl group” include a dimethylaminocarbonyl group, a methylethylaminocarbonyl group, a diisopropylaminocarbonyl group, a methylheptylaminocarbonyl group, and a dibutylaminocarbonyl group.

Examples of the term of “(C2-C6 dialkylamino)carbonyl group” include a dimethylaminocarbonyl group, a methylethylaminocarbonyl group, and a diisopropylaminocarbonyl group.

Examples of the term of “alicyclic hydrocarbon group” include cycloalkyl groups or cycloalkenyl groups.

Examples of the term of “cycloalkyl group” include a cyclopropyl group, a cyclobutyl group, a cyclopentyl group, a cyclohexyl group, a bicyclo[6.5.0]nonyl group, and a bicyclo[6.6.0]decyl group.

Examples of the term of “cycloalkenyl group” include a cyclopentenyl group and a cyclohexenyl group. Also, said cycloalkenyl group may be fused to benzene ring(s), and examples thereof include an indanyl group and a tetrahydronaphthyl group.

Examples of the term of “aryl group” include a phenyl group and a naphthyl group.

Examples of the term of “aromatic heterocyclic group” include 5 membered aromatic heterocyclic groups such as a pyrrolyl group, a furanyl group, a thienyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, and a thiadiazolyl group; 6 membered aromatic heterocyclic groups such as a pyridyl group, a pyridazinyl group, a pyrimidinyl group, a pyrazinyl group, a triazinyl group, and a tetrazinyl group; 9 membered aromatic heterocyclic groups such as an indazolyl group, an indolizinyl group, and an imidazopyridyl group; and 10 membered aromatic heterocyclic groups such as a quinolyl group, an isoquinolyl group, a quinazolinyl group, a naphthyridinyl group, and a benzopyranyl group.

Examples of the term of “nonaromatic heterocyclic group” include an aziridinyl group, an oxiranyl group, a thiiranyl group, an azetidinyl group, an oxetanyl group, a thietanyl group, a pyrrolidinyl group, a tetrahydrofuranyl group, a tetrahydrothienyl group, a pyrazolinyl group, a pyrazolidinyl group, an imidazolinyl group, an imidazolidinyl group, an oxazolinyl group, a thiazolinyl group, an oxazolidinyl group, a thiazolidinyl group, an isoxazolinyl group, an isoxazolidinyl group, an isothiazolinyl group, an isothiazolidinyl group, a dioxolanyl group, a dioxanyl group, a piperidyl group, a piperazinyl group, a morpholinyl group, a thiomorpholinyl group, a pyranyl group, a dihydropyranyl group, a tetrahydropyranyl group, a tetrahydrothiopyranyl group, an azepanyl group, an oxepanyl group, a thiepanyl group, a dihydrobenzofuranyl group, a 1,3-benzodioxolyl group, and a dihydrobenzopyranyl group.

The substituent Q in the present invention is described as follows.

Examples of Q include the following groups represented by Q1-1, Q1-2, Q1-3, Q1-4, Q1-5, Q1-6, Q1-7, Q1-8, Q2-1, and Q2-2.

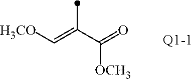

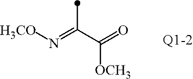

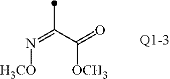

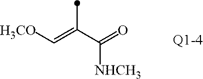

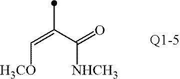

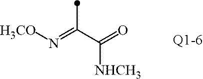

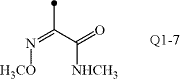

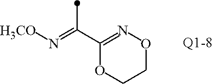

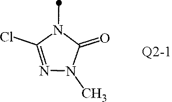

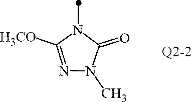

Examples of Q1 include the groups represented by Q1-1, Q1-2, Q1-3, Q1-4, Q1-5, Q1-6, Q1-7, and Q1-8.

Examples of Q2 include the groups represented by Q2-1 and Q2-2.

The substituent J in the present invention is described as follows.

Examples of J include the following groups represented by J1-1, J1-2, J1-3, J1-4, J1-5, J1-6, J1-7, J1-8, J2-1, J2-2, J2-3, J2-4, J2-5, J2-6, and J2-7.

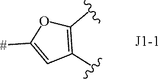

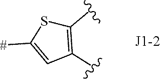

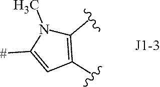

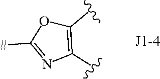

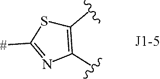

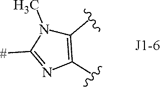

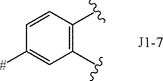

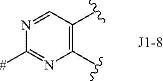

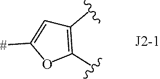

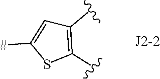

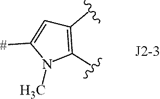

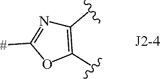

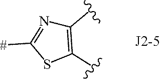

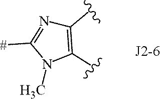

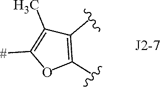

Examples of J1 include the groups represented by J1-1, J1-2, J1-3, J1-4, J1-5, J1-6, J1-7, and J1-8.

Examples of J2 include the groups represented by J2-1, J2-2, J2-3, J2-4, J2-5, J2-6, and J2-7.

For example, in the Present compound, a compound wherein J represents J1 is a compound represented by formula (I-J1), and a compound wherein J represents J1-1 is a compound represented by formula (I-J1-1).

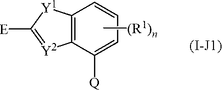

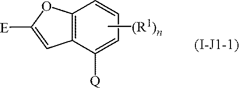

The terms used herein are described as follows.

The term of “soybean rust fungus having an amino acid substitution of F129L in a mitochondrial cytochrome b protein” represents a soybean rust fungus (scientific name: Phakopsora pachyrhizi) wherein the mitochondrial cytochrome b gene encoding the mitochondrial cytochrome protein has a mutation, and an amino acid substitution of F129L is produced as a result from said mutation, thereby a resistance to QoI fungicides is developed.

The Present compounds are QoI fungicides.

The Present compound may optionally have one or more stereoisomer(s). Examples of the stereoisomer(s) include enantiomers, diastereomers, atropisomers, and geometric isomers. The Present compound encompasses each stereoisomer and mixtures of stereoisomers at any ratio.

Examples of the term of “agriculturally acceptable salt” include acid addition salts such as hydrochloride, sulfate, nitrate, phosphate, sulfonate, acetate, and benzoate.

Aspects of the Present compound include the following compounds.

[Aspect 1] The Present compound, wherein Q represents the group represented by Q1.

[Aspect 2] The Present compound, wherein Q represents the group represented by Q2.

[Aspect 3] The Present compound, wherein Q represents the group represented by Q1-1, Q1-2, Q1-3, Q1-4, Q1-5, Q1-6, Q1-7, or Q1-8.

[Aspect 4] The Present compound, wherein Q represents the group represented by Q1-1.

[Aspect 5] The Present compound, wherein J represents the group represented by J1.

[Aspect 6] The Present compound, wherein J represents the group represented by J2.

[Aspect 7] The Present compound, wherein J represents the group represented by J1-4, J1-5, J1-7, J2-4, or J2-7.

[Aspect 8] The Present compound, wherein J represents the group represented by J1-4, J1-5, or J1-7.

[Aspect 9] The Present compound, wherein n represents 0.

[Aspect 10] The Present compound, wherein
Q represents the group represented by Q1-1, Q1-2, Q1-3, Q1-4, Q1-5, Q1-6, Q1-7, or Q1-8;
J represents the group represented by J1-4, J1-5, J1-7, J2-4, or J2-7; and
n represents 0.

[Aspect 11] The Present compound, wherein
Q represents the group represented by Q1-1;
J represents the group represented by J1-4, J1-5, or J1-7; and
n represents 0.

[Aspect 12] The Present compound, wherein E represents a C1-C6 chain hydrocarbon group optionally substituted with one or more substituent(s) selected from Group A.

[Aspect 13] The Present compound, wherein E represents a C1-C6 alkyl group optionally substituted with one or more substituent(s) selected from Group A.

[Aspect 14] The Present compound, wherein E represents a C1-C6 alkyl group optionally substituted with one or more substituent(s) selected from the group consisting of a halogen atom, a phenyl group, and a cyclopropyl group.

[Aspect 15] The Present compound, wherein E represents a methyl group, a cyclopropylmethyl group, or a benzyl group.

[Aspect 16] The Present compound, wherein E represents a C3-C10 alicyclic hydrocarbon group optionally substituted with one or more substituent(s) selected from Group B.

[Aspect 17] The Present compound, wherein E represents a C3-C6 cycloalkyl group optionally substituted with one or more substituent(s) selected from the group consisting of a C1-C3 alkyl group and a halogen atom.

[Aspect 18] The Present compound, wherein E represents a cyclopropyl group.

[Aspect 19] The Present compound, wherein E represents a 3-10 membered nonaromatic heterocyclic group optionally substituted with one or more substituent(s) selected from Group B.

[Aspect 20] The Present compound, wherein E represents a 3-10 membered nonaromatic heterocyclic group (wherein said 3-10 membered nonaromatic heterocyclic group is optionally substituted with one or more substituent(s) selected from a C1-C3 alkyl group and a halogen atom).

[Aspect 21] The Present compound, wherein E represents a C6-C10 aryl group optionally substituted with one or more substituent(s) selected from Group D.

[Aspect 22] The Present compound, wherein E represents a phenyl group optionally substituted with one or more substituent(s) selected from Group D.

[Aspect 23] The Present compound, wherein E represents a phenyl group optionally substituted with one or more halogen atom(s).

[Aspect 24] The Present compound, wherein E represents a phenyl group, a 2-chlorophenyl group, a 3-chlorophenyl group, a 4-chlorophenyl group, or 2,4-dichlorophenyl group.

[Aspect 25] The Present compound, wherein E represents a 5-10 membered aromatic heterocyclic group optionally substituted with one or more substituent(s) selected from Group D.

[Aspect 26] The Present compound, wherein E represents a 5-6 membered aromatic heterocyclic group optionally substituted with one or more substituent(s) selected from Group D.

[Aspect 27] The Present compound, wherein E represents a 5-6 membered aromatic heterocyclic group optionally substituted with one or more halogen atom(s).

[Aspect 28] The Present compound, wherein E represents a thiophen-2-yl group, a 3-chlorothiophen-2-yl group, or a furan-2-yl group.

[Aspect 29] The Present compound, wherein E represents R⁹—L¹—CH₂— or R⁴¹L²-.

[Aspect 30] The Present compound, wherein E represents a phenoxymethyl group or a (4-methylphenoxy)methyl group.

[Aspect 31] The Present compound, wherein E represents R¹⁰R¹¹C═N—O—CH₂—, R¹²O—N═C(R¹³)—C(R¹⁴)═N—O—CH₂—, R¹⁵C(O)—C(R¹⁶)═N—O—CH₂—, R¹⁷R¹⁸N—C(S)—O—CH₂—, R¹⁹N═C(R²⁰)—S—CH₂—, R²¹N═C(SR²²)—S—CH₂—, R²³O—N═C(R²⁴)—S—CH₂—, R²⁵O—N═C(SR²⁶)—S—CH₂—, R²⁷O—N═C(R²⁸)—, R²⁹R³⁰C═N—N═C(R³¹)—, R³²R³³N—N═C(R³⁴)—, R³⁵—N═C(R³⁶)—, R³⁷SC(R³⁸)═N—, R³⁹SC(SR⁴⁰)═N—, R⁴³C(O)O—, R⁴⁴OC(O)O—, R⁴⁵R⁴⁶NC(O)O—, R⁴⁷R⁴⁸NC(S)O—, R⁴⁹S(O)₂O—, R⁵⁰R⁵¹NS(O)₂O—, a cyano group, a nitro group, or a hydroxy group.

[Aspect 32] The Present compound, wherein E represents R¹⁰R¹¹C═N—O—CH₂— or R²⁷O—N═C(R²⁸)—.

[Aspect 33] The Present compound, wherein E represents a halogen atom.

[Aspect 34] The Present compound, wherein E represents a bromine atom.

[Aspect 35] The compound according to the Aspect 10, wherein E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a 3-10 membered nonaromatic heterocyclic group (wherein said 3-10 membered nonaromatic heterocyclic group is optionally substituted with one or more substituent(s) selected from a C1-C3 alkyl group and a halogen atom), a phenyl group, a 5-6 membered aromatic heterocyclic group {wherein said phenyl group and said 5-6 membered aromatic heterocyclic group are optionally substituted with one or more substituent(s) selected from Group D}, R⁹—L¹—CH₂—, R⁴¹L²-, R¹⁰R¹¹C═N—O—CH₂—, R²⁷O—N═C(R²⁸)—, or a halogen atom.

[Aspect 36] The compound according to the Aspect 11, wherein E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a 3-10 membered nonaromatic heterocyclic group (wherein said 3-10 membered nonaromatic heterocyclic group is optionally substituted with one or more substituent(s) selected from a C1-C3 alkyl group and a halogen atom), a phenyl group, a 5-6 membered aromatic heterocyclic group {wherein said phenyl group and said 5-6 membered aromatic heterocyclic group are optionally substituted with one or more substituent(s) selected from Group D}, R⁹—L¹—CH₂—, R⁴¹L²-, R¹⁰R¹¹C═N—O—CH₂—, R²⁷O—N═C(R²⁸)—, or a halogen atom.

[Aspect 37] The compound according to the Aspect 10, wherein E represents a C1-C6 alkyl group (wherein said C1-C6 alkyl group is optionally substituted with one or more substituent(s) selected from a halogen atom, a phenyl group, and a cyclopropyl group), a C3-C6 cycloalkyl group {wherein said C3-C6 cycloalkyl group is optionally substituted with one or more substituent(s) selected from the group consisting of a C1-C3 alkyl group and a halogen atom}, a 3-10 membered nonaromatic heterocyclic group (wherein said 3-10 membered nonaromatic heterocyclic group is optionally substituted with one or more substituent(s) selected from a C1-C3 alkyl group and a halogen atom), a phenyl group, a 5-6 membered aromatic heterocyclic group {wherein said phenyl group and said 5-6 membered aromatic heterocyclic group are optionally substituted with one or more halogen atom(s)}, R⁹—L¹—CH₂—, R⁴¹L²-, R¹⁰R¹¹C═N—O—CH₂—, R²⁷O—N═C(R²⁸)—, or a halogen atom.

[Aspect 38] The compound according to the Aspect 10, wherein E represents a C1-C6 alkyl group (wherein said C1-C6 alkyl group is optionally substituted with one or more substituent(s) selected from a halogen atom, a phenyl group, and a cyclopropyl group), a C3-C6 cycloalkyl group {wherein said C3-C6 cycloalkyl group is optionally substituted with one or more substituent(s) selected from the group consisting of a C1-C3 alkyl group and a halogen atom}, a 3-10 membered nonaromatic heterocyclic group (wherein said 3-10 membered nonaromatic heterocyclic group is optionally substituted with one or more substituent(s) selected from a C1-C3 alkyl group and a halogen atom), a phenyl group, a 5-6 membered aromatic heterocyclic group {wherein said phenyl group and said 5-6 membered aromatic heterocyclic group are optionally substituted with one or more halogen atom(s)}, R⁹—L¹—CH₂—, R⁴¹L²-, R¹⁰R¹¹C═N—O—CH₂—, R²⁷O—N═C(R²⁸)—, or a halogen atom.

[Aspect 39] The compound according to the Aspect 11, wherein E represents a methyl group, a cyclopropylmethyl group, a benzyl group, a cyclopropyl group, a phenyl group, a 2-chlorophenyl group, a 3-chlorophenyl group, a 4-chlorophenyl group, a 2,4-dichlorophenyl group, a thiophen-2-yl group, a 3-chlorothiophen-2-yl group, a furan-2-yl group, a 4-methylphenoxymethyl group, a phenoxymethyl group, or a bromine atom.

[Aspect 40] The compound according to the Aspect 11, wherein E represents a phenyl group optionally substituted with one or more halogen atom(s).

[Aspect 41] The compound according to the Aspect 11, wherein E represents a phenyl group.

Next, Production methods for the Present compounds disclosed herein are described.

### Production Method A

A compound represented by formula (A1) (hereinafter referred to as “Compound (A1)”) may be prepared by reacting a compound represented by formula (B1) (hereinafter referred to as “Compound (B1)”) with a compound represented by formula (M1) (hereinafter referred to as “Compound (M1)”) in the presence of a palladium catalyst and a base.

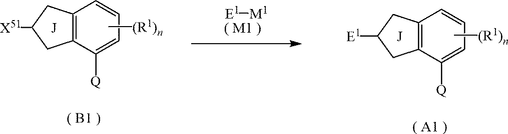

[wherein E¹ represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D}; M¹ represents B(OH)₂ or a 4,4,5,5-tetramethyl-1,3,2-dioxaborolan-2-yl group; X⁵¹ represents a leaving group such as a chlorine atom, a bromine atom, an iodine atom, and a triflyloxy group; and the other symbols are the same as defined above.]

The reaction is usually carried out in a solvent. Examples of the solvent to be used in the reaction include hydrocarbons such as hexane, toluene, and xylene (hereinafter collectively referred to as “hydrocarbons”); ethers such as methyl tert-butyl ether (hereinafter referred to as “MTBE”), tetrahydrofuran (hereinafter referred to as “THF”), and dimethoxyethane (hereinafter collectively referred to as “ethers”); halogenated hydrocarbons such as chloroform and chlorobenzene (hereinafter collectively referred to as “halogenated hydrocarbons”); amides such as dimethylformamide (hereinafter referred to as “DMF”) and N-methylpyrrolidone (hereinafter collectively referred to as “amides”); esters such as methyl acetate and ethyl acetate (hereinafter collectively referred to as “esters”); nitriles such as acetonitrile and propionitrile (hereinafter collectively referred to as “nitriles”); water; and mixtures of two or more of them.

Examples of the palladium catalyst to be used in the reaction include [1,1′-bis(diphenylphosphino)ferrocene]palladium(II) dichloride.

Examples of the base to be used in the reaction include organic bases such as triethylamine and pyridine (hereinafter collectively referred to as “organic bases”); alkali metal carbonates such as sodium carbonate and potassium carbonate (hereinafter collectively referred to as “alkali metal carbonates”); alkali metal hydrogen carbonates such as sodium hydrogen carbonate and potassium hydrogen carbonate (hereinafter collectively referred to as “alkali metal hydrogen carbonates”); sodium fluoride; and tripotassium phosphate.

In the reaction, the Compound (M1) is usually used at a ratio of 1 to 10 mol, the palladium catalyst is usually used at a ratio of 0.01 to 1 mol, and the base is usually used at a ratio of 1 to 10 mol, relative to 1 mol of the Compound (B1).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A1).

The Compound (B1) and the Compound (M1) are known or may be prepared according to known methods.

### Production Method B

The Compound (A1) may also be prepared by reacting a compound represented by formula (B2) (hereinafter referred to as “Compound (B2)”) with a compound represented by formula (M2) (hereinafter referred to as “Compound (M2)”) in the presence of a palladium catalyst and a base.

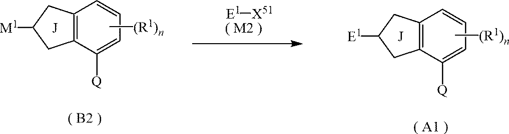

[wherein the symbols are the same as defined above.]

The reaction may be carried out according to the Production method A by using the Compound (M2) instead of the Compound (B1), and using the Compound (B2) instead of the Compound (M1).

The Compound (B2) and the Compound (M2) are known or may be prepared according to known methods.

### Production Method C

A compound represented by formula (A2) (hereinafter referred to as “Compound (A2)”) may be prepared by reacting the Compound (B1) with a compound represented by formula (M3) (hereinafter referred to as “Compound (M3)”) in the presence of a metal catalyst and a base.

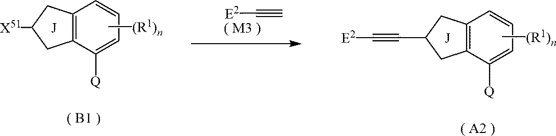

[wherein E² represents a C1-C4 chain hydrocarbon group optionally substituted with one or more substituent(s) selected from Group A; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the metal catalyst to be used in the reaction include bis(triphenylphosphine)palladium(II) dichloride (hereinafter referred to as “PdCl₂ (PPh₃)₂”) and copper(I) iodide.

Examples of the base to be used in the reaction include organic bases.

In the reaction, the Compound (M3) is usually used at a ratio of 1 to 10 mol, the metal catalyst is usually used at a ratio of 0.01 to 1 mol, and the base is usually used at a ratio of 1 to 10 mol, relative to 1 mol of the Compound (B1).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A2).

The Compound (M3) is known or may be prepared according to known method(s).

### Production Method D

A compound represented by formula (A3) (hereinafter referred to as “Compound (A3)”) may be prepared by reacting a compound represented by formula (B3) (hereinafter referred to as “Compound (B3)”) with a compound represented by formula (M4) (hereinafter referred to as “Compound (M4)”) or a salt thereof.

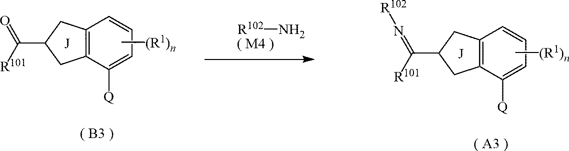

[wherein the combination of R¹⁰¹ and R¹⁰² represents a combination wherein R¹⁰¹ represents R²⁸ and R¹⁰² represents R²⁷O-, a combination wherein R¹⁰¹ represents R³¹ and R¹⁰² represents R²⁹R³⁰C═N—, a combination wherein R¹⁰¹ represents R³⁴ and R¹⁰² represents R³²R³³N-, or a combination wherein R¹⁰¹ represents R³⁶ and R¹⁰² represents R³⁵; and the other symbols are the same as defined above.]

Examples of the salt of the Compound (M4) include hydrochloride and sulfate.

The reaction is usually carried out in a solvent. Examples of the solvent to be used in the reaction include hydrocarbons; ethers; halogenated hydrocarbons; amides; esters; nitriles; alcohol such as methanol and ethanol (hereinafter collectively referred to as “alcohols”); and mixtures of two or more of them.

In the reaction, a base may be used as needed.

Examples of the base to be used in the reaction include organic bases, alkali metal carbonates, alkali metal hydrogen carbonates, sodium hydride, and tripotassium phosphate.

When a base is used in the reaction, the base is usually used at a ratio of 1 to 10 mol relative to 1 mol of the Compound (B3).

In the reaction, the Compound (M4) is usually used at a ratio of 1 to 10 mol relative to 1 mol of the Compound (B3).

The reaction temperature is usually within the range of 0 to 150° C. The reaction time is usually within the range of 0.1 to 120 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A3).

The Compound (M4) is known or may be prepared according to known method(s).

### Production Method E

A compound represented by formula (A4) (hereinafter referred to as “Compound (A4)”) may be prepared by reacting a compound represented by formula (B4) (hereinafter referred to as “Compound (B4)”) with a compound represented by formula (M5) (hereinafter referred to as “Compound (M5)”) in the presence of a base.

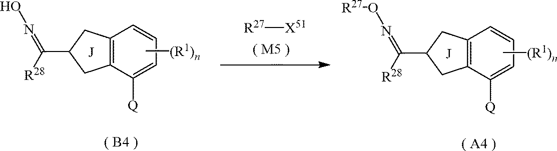

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B4).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B4).

In the reaction, the Compound (M5) is usually used at a ratio of 1 to 10 mol, and the base is usually used at a ratio of 1 to 10 mol, relative to 1 mol of the Compound (B4).

The reaction temperature is usually within the range of -20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A4).

The Compound (M5) is known or may be prepared according to known method(s).

### Production Method F

A compound represented by formula (A5) (hereinafter referred to as “Compound (A5)”) may be prepared by reacting a compound represented by formula (B5) (hereinafter referred to as “Compound (B5)”) with a compound represented by formula (M6) (hereinafter referred to as “Compound (M6)”) in the presence of a base.

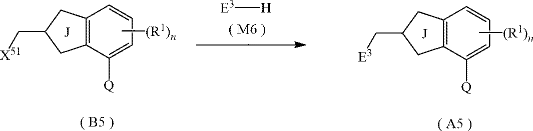

[wherein E³ represents R⁹—L¹—, R¹⁰R¹¹C═N—O—, R¹²O—N═C(R¹³)—C(R¹⁴)═N—O—, R¹⁵C(O)—C(R¹⁶)═N—O—, R¹⁷R¹⁸N—C(S)—O—, R¹⁹N═C(R²⁰)—S—, R²¹N═C(SR²²)—S—, R²³O—N═C(R²⁴)—S—, or R²⁵O—N═C(SR²⁶)—S—; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, the Compound (M6) is usually used at a ratio of 1 to 10 mol, and the base is usually used at a ratio of 1 to 10 mol, relative to 1 mol of the Compound (B5).

The reaction temperature is usually within the range of -20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A5).

The Compound (M6) is known or may be prepared according to known method(s).

### Production Method G

A compound represented by formula (A6) (hereinafter referred to as “Compound (A6)”) may be prepared by reacting a compound represented by formula (B6) (hereinafter referred to as “Compound (B6)”) with a compound represented by formula (M7) (hereinafter referred to as “Compound (M7)”) in the presence of a phosphine and an azodiester.

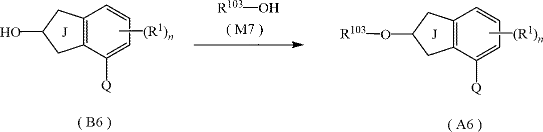

[wherein R¹⁰³ represents a C1-C6 chain hydrocarbon group optionally substituted with one or more substituent(s) selected from Group A or a C3-C10 alicyclic hydrocarbon group optionally substituted with one or more substituent(s) selected from Group B; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the phosphine include triphenylphosphine and trimethylphosphine.

Examples of the azodiester include diethyl azodicarboxylate, diisopropyl azodicarboxylate, and bis(2-methoxyethyl) azodicarboxylate.

In the reaction, the Compound (M7) is usually used at a ratio of 1 to 10 mol, the phosphine is usually used at a ratio of 1 to 10 mol, and the azodiester is usually used at a ratio of 1 to 10 mol, relative to 1 mol of the Compound (B6).

The reaction temperature is usually within the range of 0 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A6).

The Compound (M7) is known or may be prepared according to known method(s).

### Production Method H

A compound represented by formula (A7) (hereinafter referred to as “Compound (A7)”) may be prepared by reacting a compound represented by formula (B7) (hereinafter referred to as “Compound (B7)”) with a compound represented by formula (M8) (hereinafter referred to as “Compound (M8)”) in the presence of a base.

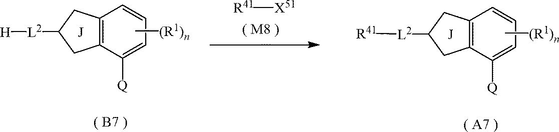

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B7).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B7).

In the reaction, the Compound (M8) is usually used at a ratio of 1 to 10 mol, and the base is usually used at a ratio of 1 to 10 mol, relative to 1 mol of the Compound (B7).

The reaction temperature is usually within the range of -20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A7).

The Compound (B7) and the Compound (M8) are known or may be prepared according to known method(s).

### Production Method I

The Compound (A7) may also be prepared by reacting the Compound (B1) with a compound represented by formula (M9) (hereinafter referred to as “Compound (M9)”) in the presence of a base.

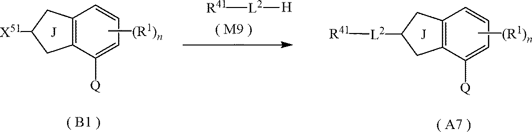

[wherein the symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, a metal catalyst and/or a ligand may be used as needed.

Examples of the metal catalyst include copper catalysts such as copper(I) iodide, copper(I) bromide, copper(I) chloride, copper(I) oxide, copper(I) trifluoromethanesulfonate benzene complex, tetrakis(acetonitrile)copper(I) hexafluorophosphate, and copper(I) 2-thiophenecarboxylate; and nickel catalysts such as bis(cyclooctadiene)nickel(0) and nickel(II) chloride. When a metal catalyst is used in the reaction, the metal catalyst is usually used at a ratio of 0.01 to 1 mol relative to 1 mol of the Compound (B1).

Examples of the ligand include triphenylphosphine, Xantphos, 2,2′-bis(diphenylphosphino)-1,1′-binaphthyl, 1,1′-bis(diphenylphosphino)ferrocene, 2-dicyclohexylphosphino-2′,4′,6′-triisopropylbiphenyl, 2-dicyclohexylphosphino-2′,6′-dimethoxybiphenyl, 1,2-bis(diphenylphosphino)ethane, 2,2′-bipyridine, 2-aminoethanol, 8-hydroxyquinoline, 1,10-phenanthroline, trans-1,2-cyclohexanediamine, trans-N,N′-dimethylcyclohexane-1,2-diamine, N,N′-dimethylethylenediamine, and N,N-dimethylglycine hydrochloride. When a ligand is used in the reaction, the ligand is usually used at a ratio of 0.01 to 1 mol relative to 1 mol of the Compound (B1).

In the reaction, the Compound (M9) is usually used at a ratio of 1 to 10 mol, and the base is usually used at a ratio of 1 to 10 mol, relative to 1 mol of the Compound (B1).

The reaction temperature is usually within the range of -20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A7).

The Compound (M9) is known or may be prepared according to known method(s).

### Production Method J

A compound represented by formula (A8) (hereinafter referred to as “Compound (A8)”) may be prepared by reacting the Compound (B6) with a compound represented by formula (M10) (hereinafter referred to as “Compound (M10)”) in the presence of a base.

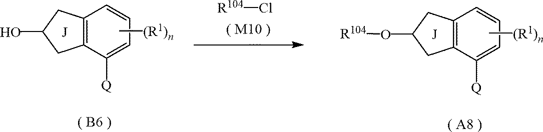

[wherein R¹⁰⁴ represents R⁴³C(O)—, R⁴⁴OC(O)—, R⁴⁵R⁴⁶NC(O)—, R⁴⁷R⁴⁸NC(S)—, R⁴⁹S(O)₂—, or R⁵⁰R⁵¹NS(O)₂—; and the other symbols are the same as defined above.]

The reaction is usually carried out in a solvent. Examples of the solvent to be used in the reaction include hydrocarbons, ethers, halogenated hydrocarbons, amides, esters, nitriles, and mixtures of two or more of them.

Examples of the base include organic bases, alkali metal carbonates, alkali metal hydrogen carbonates, sodium hydride, and tripotassium phosphate.

In the reaction, the Compound (M10) is usually used at a ratio of 1 to 10 mol, and the base is usually used at a ratio of 1 to 10 mol, relative to 1 mol of the Compound (B6).

The reaction temperature is usually within the range of -78 to 100° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A8).

The Compound (M10) is known or may be prepared according to known method(s).

### Production Method K

A compound represented by formula (A9) (hereinafter referred to as “Compound (A9)”) may be prepared by carrying out a step of reacting a compound represented by formula (B8) (hereinafter referred to as “Compound (B8)”) with a compound represented by formula (M11) (hereinafter referred to as “Compound (M11)”) in the presence of a base to give a compound represented by formula (B9) (hereinafter referred to as “Compound (B9)”) (hereinafter referred to as “Step (K-1)”), and a step of reacting the Compound (B9) with a compound represented by formula (M12) (hereinafter referred to as “Compound (M12)”) in the presence of a base (hereinafter referred to as “Step (K-2)”).

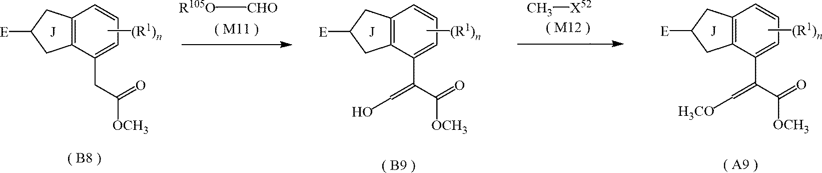

[wherein R¹⁰⁵ represents a C1-C4 alkyl group; X⁵² represents an iodine atom, a methoxysulfonyl group, a mesyloxy group, or a tosyloxy group; and the other symbols are the same as defined above.]

The Step (K-1) is usually carried out in a solvent. Examples of the solvent to be used in the reaction include ethers, amides, and mixtures of two or more of them.

Examples of the base to be used in the reaction include alkali metal hydrides such as sodium hydride.

In the reaction, the Compound (M11) is usually used at a ratio of 1 mol to 10 mol, the base is usually used at a ratio of 0.5 mol to 5 mol, relative to 1 mol of the Compound (B8).

The reaction time is usually within the range of 5 minutes to 72 hours. The reaction temperature is usually within the range of -20° C. to 100° C.

When the reaction is completed, the reaction mixture may be subjected to a work-up such as adding water to the reaction mixture, extracting the resulting mixture with organic solvent(s), and drying and/or concentrating the resulting organic layer to give the Compound (B9).

The Compound (B8) and the Compound (M11) are commercially available compounds or may be prepared according to known methods.

The Step (K-2) is usually carried out in a solvent. Examples of the solvent to be used in the reaction include hydrocarbons, ethers, halogenated hydrocarbons, amides, esters, nitriles, and mixtures of two or more of them.

Examples of the base to be used in the reaction include organic bases, alkali metal carbonates, alkali metal hydrogen carbonates, sodium hydride, and mixtures of two or more of them.

In the reaction, the Compound (M12) is usually used at a ratio of 1 to 10 mol, and the base is usually used at a ratio of 1 to 20 mol, relative to 1 mol of the Compound (B9).

The reaction temperature is usually within the range of -20 to 100° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A9).

The Compound (M12) is known or may be prepared according to known method(s).

### Production Method L

A compound represented by formula (A10) (hereinafter referred to as “Compound (A10)”) may be prepared by carrying out a step of reacting the Compound (B8) with a compound represented by formula (M13) (hereinafter referred to as “Compound (M13)”) in the presence of a base to give a compound represented by formula (B10) (hereinafter referred to as “Compound (B10)”) (hereinafter referred to as “Step (L-1)”), and a step of reacting the Compound (B10) with the Compound (M12) in the presence of a base (hereinafter referred to as “Step (L-2)”).

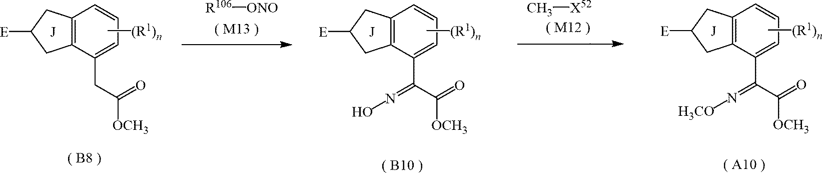

[wherein R¹⁰⁶ represents a tert-butyl group or an isopentyl group; and the other symbols are the same as defined above.]

The Step (L-1) is usually carried out in a solvent. Examples of the solvent to be used in the reaction include ethers, amides, alcohols, and mixtures of two or more of them.

Examples of the base to be used in the reaction include sodium hydride; and alkali metal alkoxides such as sodium methoxide, sodium ethoxide, and potassium t-butoxide.

In the reaction, the Compound (M13) is usually used at a ratio of 1 mol to 10 mol, and the base is usually used at a ratio of 1 mol to 5 mol, relative to 1 mol of the Compound (B8).

The reaction time is usually within the range of 5 minutes to 72 hours. The reaction temperature is usually within the range of -20° C. to 100° C.

When the reaction is completed, the reaction mixture may be subjected to a work-up such as adding water to the reaction mixture, extracting the resulting mixture with organic solvent(s), and drying and/or concentrating the resulting organic layer to give the Compound (B10).

The Compound (M13) is a commercially available compound.

The Step (L-2) may be carried out according to the Step (K-2) of the Production method K by using the Compound (B10) instead of the Compound (B9).

### Production Method M

A compound represented by formula (A12) (hereinafter referred to as “Compound (A12)”) may be prepared by reacting a compound represented by formula (A11) (hereinafter referred to as “Compound (A11)”) with methylamine.

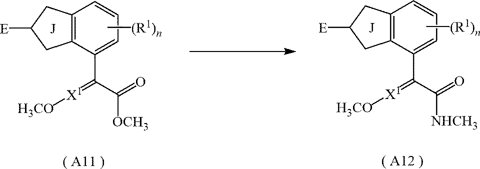

[wherein the symbols are the same as defined above.]

The reaction is usually carried out in a solvent. Examples of the solvent to be used in the reaction include alcohols, hydrocarbons, ethers, halogenated hydrocarbons, amides, esters, nitriles, and mixtures of two or more of them.

In the reaction, a base may be used as needed. Examples of the base to be used in the reaction include organic bases; alkali metal carbonates; alkali metal hydroxides such as sodium hydroxide and potassium hydroxide (hereinafter collectively referred to as “alkali metal hydroxides”); and sodium hydride. When a base is used in the reaction, the base is usually used at a ratio of 0.1 to 10 mol relative to 1 mol of the Compound (A11).

The methylamine is usually used as a solution. Examples of the solution of methylamine include a methanol solution and an aqueous solution.

In the reaction, the methylamine is usually used at a ratio of 1 to 100 mol relative to 1 mol of the Compound (A11).

The reaction temperature is usually within the range of -20 to 60° C. The reaction time is usually within the range of 0.1 to 120 hour(s).

When the reaction is completed, the reaction mixture may be subjected to a work-up such as mixing the reaction mixture with water, then extracting the resulting mixture with organic solvent(s), and drying and/or concentrating the resulting organic layer to isolate the Compound (A12).

### Production Method N

A compound represented by formula (A13) (hereinafter referred to as “Compound (A13)”) may be prepared by carrying out a step of reacting the Compound (A10) with hydroxylamine in the presence of a base to give a compound represented by formula (B11) (hereinafter referred to as “Compound (B11)”) (hereinafter referred to as “Step (N-1)”), and a step of reacting the Compound (B11) with a compound represented by formula (M14) (hereinafter referred to as “Compound (M14)”) in the presence of a base (hereinafter referred to as “Step (N-2)”).

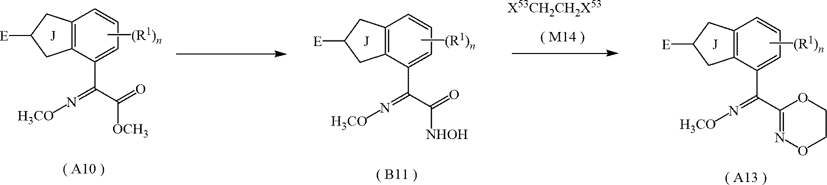

[wherein X⁵³ represents a chlorine atom, a bromine atom, or an iodine atom; and the other symbols are the same as defined above.]

The Step (N-1) may be carried out according to the Production method M by using the Compound (A10) instead of the Compound (A11), and using hydroxylamine instead of methylamine.

The Step (N-2) is usually carried out in a solvent. Examples of the solvent to be used in the reaction include hydrocarbons, ethers, halogenated hydrocarbons, amides, esters, nitriles, water, and mixtures of two or more of them.

Examples of the base include organic bases, alkali metal carbonates, alkali metal hydrogen carbonates, sodium hydride, and tripotassium phosphate.

In the reaction, the Compound (M14) is usually used at a ratio of 1 to 10 mol, and the base is usually used at a ratio of 1 to 10 mol, relative to 1 mol of the Compound (B11).

The reaction temperature is usually within the range of -20 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A13).

The Compound (M14) is known or may be prepared according to known method(s).

### Production Method O

A compound represented by formula (A15) (hereinafter referred to as “Compound (A15)”) may be prepared by carrying out a step of reacting a compound represented by formula (B12) (hereinafter referred to as “Compound (B12)”) with triphosgene to give a compound represented by formula (B13) (hereinafter referred to as “Compound (B13)”)

(hereinafter referred to as “Step (O-1)”), a step of reacting the Compound (B13) with N,N-dimethylhydrazine to give a compound represented by formula (B14) (hereinafter referred to as “Compound (B14)”) (hereinafter referred to as “Step (O-2)”), a step of reacting the Compound (B14) with triphosgene to give a compound represented by formula (A14) (hereinafter referred to as “Compound (A14)”) (hereinafter referred to as “Step (O-3)”), and a step of reacting the Compound (A14) with a compound represented by formula (M15) (hereinafter referred to as “Compound (M15)”) in the presence of a base (hereinafter referred to as “Step (O-4) ”) .

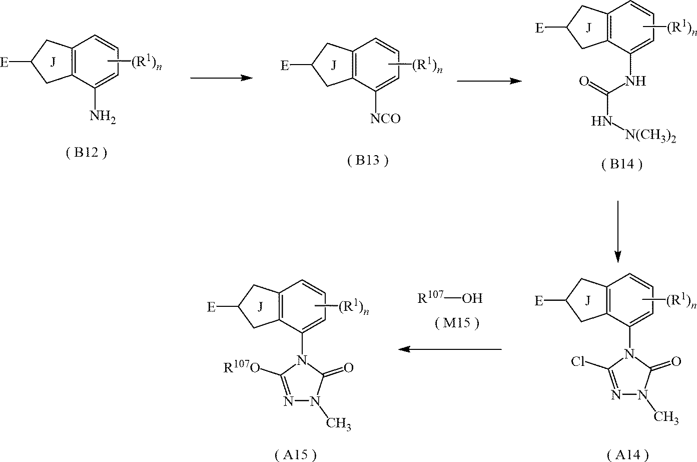

[wherein R¹⁰⁷ is a C1-C3 chain hydrocarbon group optionally substituted with one or more halogen atom(s); and the other symbols are the same as defined above.]

The Step (O-1) is usually carried out in a solvent. Examples of the solvent to be used in the reaction include hydrocarbons, halogenated hydrocarbons, ethers, and mixtures of two or more of them.

In the reaction, the triphosgene is usually used at a ratio of 0.3 mol to 5 mol relative to 1 mol of the Compound (B12) .

The reaction time is usually within the range of 5 minutes to 72 hours. The reaction temperature is usually within the range of 0° C. to 150° C.

When the reaction is completed, the reaction mixture may be subjected to a work-up such as concentrating the reaction mixture to give the Compound (B13).

The Compound (B12) is a commercially available compound or may be prepared according to known method(s).

The Step (O-2) is usually carried out in a solvent. Examples of the solvent to be used in the reaction include hydrocarbons, ethers, amides, and mixtures of two or more of them.

In the reaction, the N,N-dimethylhydrazine is usually used at a ratio of 0.8 mol to 5 mol relative to 1 mol of the Compound (B13).

The reaction time is usually within the range of 5 minutes to 72 hours. The reaction temperature is usually within the range of -20° C. to 100° C.

When the reaction is completed, the reaction mixture may be subjected to a work-up such as collecting the precipitated solids by filtration, or adding water to the reaction mixture, extracting the resulting mixture with organic solvent(s), and drying and/or concentrating the resulting organic layer to give the Compound (B14).

The Step (O-3) is usually carried out in a solvent. Examples of the solvent to be used in the reaction include hydrocarbons, ethers, halogenated hydrocarbons, and mixtures of two or more of them.

In the reaction, the triphosgene is usually used at a ratio of 1 mol to 10 mol relative to 1 mol of the Compound (B14) .

The reaction time is usually within the range of 5 minutes to 72 hours. The reaction temperature is usually within the range of 0° C. to 150° C.

When the reaction is completed, the reaction mixture may be subjected to a work-up such as adding water to the reaction mixture, extracting the resulting mixture with organic solvent(s), and drying and/or concentrating the resulting organic layer to give the Compound (A14).

The Step (O-4) is usually carried out in a solvent. Examples of the solvent to be used in the reaction include hydrocarbons, ethers, halogenated hydrocarbons, amides, esters, nitriles, and mixtures of two or more of them.

Examples of the base to be used in the reaction include organic bases, alkali metal carbonates, alkali metal hydrogen carbonates, sodium hydride, and mixtures of two or more of them.

In the reaction, the Compound (M15) is usually used at a ratio of 1 to 10 mol, and the base is usually used at a ratio of 1 to 20 mol, relative to 1 mol of the Compound (A14).

The reaction temperature is usually within the range of -20 to 100° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (A15).

The Compound (M15) is a commercially available compound or may be prepared according to known method(s).

### Production Method P

The Compound (A9) may be prepared by reacting a compound represented by formula (B15) (hereinafter referred to as “Compound (B15)”) with a compound represented by formula (M16) (hereinafter referred to as “Compound (M16)”) in the presence of a palladium catalyst and a base.

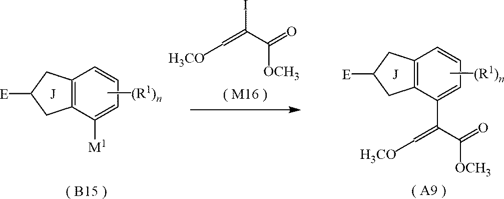

[wherein the symbols are the same as defined above.]

The Compound (A9) may be prepared according to the method described in the Production method A by using the Compound (B15) instead of the Compound (M1), and using the Compound (M16) instead of the Compound (B1).

The Compound (M16) is a known compound.

### Production Method Q

A compound represented by formula (A17) (hereinafter referred to as “Compound (A17)”) may be prepared by carrying out a step of reacting a compound represented by formula (B16) (hereinafter referred to as “Compound (B16)”) with a compound represented by formula (M17) (hereinafter referred to as “Compound (M17)”) to give a compound represented by formula (B17) (hereinafter referred to as “Compound (B17)”) (hereinafter referred to as “Step (Q-1)”), and a step of subjecting the Compound (B17) to intramolecular condensation (hereinafter referred to as “Step (Q-2)”).

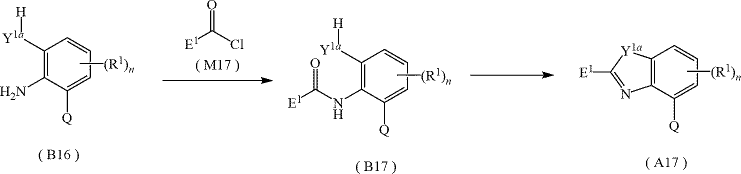

[wherein Y^{1a} represents an oxygen atom, a sulfur atom, or —N(R²)—; and the other symbols are the same as defined above.]

The Step (Q-1) is usually carried out in a solvent. Examples of the solvent to be used in the reaction include hydrocarbons, ethers, halogenated hydrocarbons, amides, esters, nitriles, and mixtures of two or more of them.

In the reaction, the Compound (M17) is usually used at a ratio of 0.8 mol to 5 mol relative to 1 mol of the Compound (B16).

In the reaction, a base may be used as needed.

Examples of the base to be used in the reaction include organic bases and alkali metal carbonates. The base is usually used at a ratio of 0.05 to 5 mol relative to 1 mol of the Compound (B16).

The reaction time is usually within the range of 5 minutes to 72 hours. The reaction temperature is usually within the range of -20° C. to 100° C.

When the reaction is completed, the reaction mixture may be subjected to a work-up such as collecting the precipitated solids by filtration, or adding water to the reaction mixture, extracting the resulting mixture with organic solvent(s), and drying and/or concentrating the resulting organic layer to give the Compound (B17).

The Compound (B16) and the Compound (M17) are known or may be prepared according to known method(s).

The Step (Q-2) is usually carried out in a solvent. Examples of the solvent to be used in the reaction include hydrocarbons, ethers, halogenated hydrocarbons, amides, esters, nitriles, and mixtures of two or more of them.

In the reaction, an acid or a base may be used as needed.

Examples of the acid to be used in the reaction include sulfonic acids such as p-toluenesulfonic acid, carboxylic acids such as acetic acid, and polyphosphoric acid.

Examples of the base to be used in the reaction include organic bases and alkali metal carbonates.

In the reaction, when an acid is used, the acid is usually used at a ratio of 0.1 mol to 5 mol, and when a base is used, the base is usually used at a ratio of 1 mol to 5 mol, relative to 1 mol of the Compound (B17).

The reaction time is usually within the range of 5 minutes to 72 hours. The reaction temperature is usually within the range of 50° C. to 150° C.

When the reaction is completed, the reaction mixture may be subjected to a work-up such as collecting the precipitated solids by filtration, or adding water to the reaction mixture, extracting the resulting mixture with organic solvent(s), and drying and/or concentrating the resulting organic layer to give the Compound (A17).

### Production Method R

A compound represented by formula (A18) (hereinafter referred to as “Compound (A18)”) may be prepared by carrying out a step of reacting a compound represented by formula (B18) (hereinafter referred to as “Compound (B18)”) with the Compound (M17) to give a compound represented by formula (B19) (hereinafter referred to as “Compound (B19)”) (hereinafter referred to as “Step (R-1)”), and a step of subjecting the Compound (B19) to intramolecular condensation (hereinafter referred to as “Step (R-2)”).

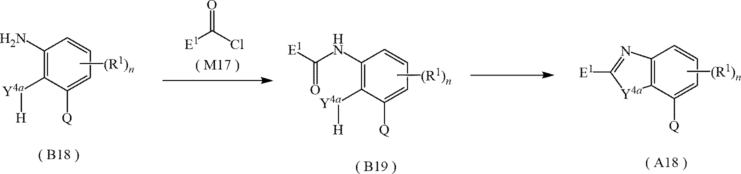

[wherein Y^{4a} represents an oxygen atom, a sulfur atom, or —N(R⁸)—; and the other symbols are the same as defined above.]

The Step (R-1) may be carried out according to the method described in the Step (Q-1) of the Production method Q by using the Compound (B18) instead of the Compound (B16).

The Compound (M18) is known or may be prepared according to known method(s).

The Step (R-2) may be carried out according to the method described in the Step (Q-2) of the Production method Q by using the Compound (B19) instead of the Compound (B17).

### Production Method S

The N-oxide of the compound represented by formula (I) may be prepared by reacting the compound represented by formula (I) with an oxidizing agent.

The reaction may be carried out according to the method described in, for example, U.S. Pat. Application Publication No. 2018/0009778 or WO 2016/121970 pamphlet.

### Reference Production Method A

A compound represented by formula (B65) (hereinafter referred to as “Compound (B65)”) may be prepared by reacting the Compound (B1) with bis(pinacolato)diboron in the presence of a base and a palladium catalyst.

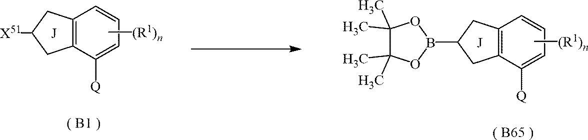

[wherein the symbols are the same as defined above.]

The reaction is usually carried out in a solvent. Examples of the solvent to be used in the reaction include hydrocarbons; ethers; halogenated hydrocarbons; amides; esters; sulfoxides such as dimethylsulfoxide (hereinafter referred to as “DMSO”) (hereinafter collectively referred to as “sulfoxides”); nitriles; and mixtures of two or more of them.

Examples of the base to be used in the reaction include organic bases, alkali metal carbonates, alkali metal hydrogen carbonates, and tripotassium phosphate.

Examples of the palladium catalyst include [1,1′-bis(diphenylphosphino)ferrocene]palladium(II) dichloride.

In the reaction, the bis(pinacolato)diboron is usually used at a ratio of 1 to 5 mol, the base is usually used at a ratio of 1 to 5 mol, and the palladium catalyst is usually used at a ratio of 0.01 to 0.5 mol, relative to 1 mol of the Compound (B1).

The reaction temperature is usually within the range of 0 to 150° C. The reaction time is usually within the range of 0.1 to 48 hour(s).

When the reaction is completed, the reaction mixture may be subjected to a work-up such as adding water to the reaction mixture, extracting the resulting mixture with organic solvent(s), and drying and/or concentrating the resulting organic layer to isolate the Compound (B65).

### Reference Production Method B

A compound represented by formula (B66) (hereinafter referred to as “Compound (B66)”) may be prepared by reacting the Compound (B1) with a compound represented by formula (M18) (hereinafter referred to as “Compound (M18)”).

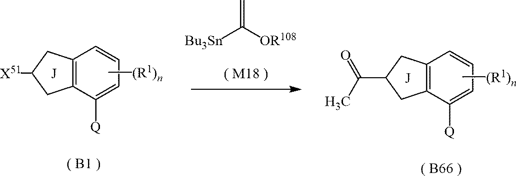

[wherein R¹⁰⁸ represents a methyl group or an ethyl group; and the other symbols are the same as defined above.]

The reaction may be carried out according to the method described in, for example, WO 2016/123253 pamphlet.

The Compound (M18) is a known compound.

### Reference Production Method C

A compound represented by formula (B67) (hereinafter referred to as “Compound (B67)”) may be prepared by reacting the Compound (B66) with hydroxylamine or a salt thereof.

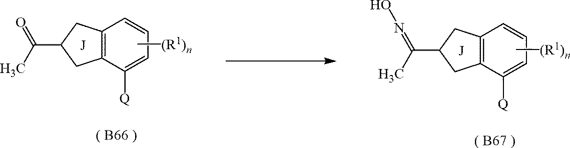

[wherein the symbols are the same as defined above.]

Examples of the salt of hydroxylamine include hydrochloride and sulfate.

The reaction may be carried out according to the Production method D by using the Compound (B66) instead of the Compound (B3), and using hydroxylamine or a salt thereof instead of the Compound (M4).

### Reference Production Method D

A compound represented by formula (B69) (hereinafter referred to as “Compound (B69)”) may be prepared by carrying out a step of reacting the Compound (B1) with N-formylsaccharin in the presence of a palladium catalyst, a ligand, triethylsilane, and a base to give a compound represented by formula (B68) (hereinafter referred to as “Compound (B68)”) (hereinafter referred to as “Step (d-1)”), a step of reacting the Compound (B68) with sodium borohydride to give the Compound (B5) (hereinafter referred to as “Step (d-2)”), and a step of reacting the Compound (B5) with carbon tetrachloride, carbon tetrabromide, or iodine in the presence of triphenylphosphine (hereinafter referred to as “Step (d-3)”).

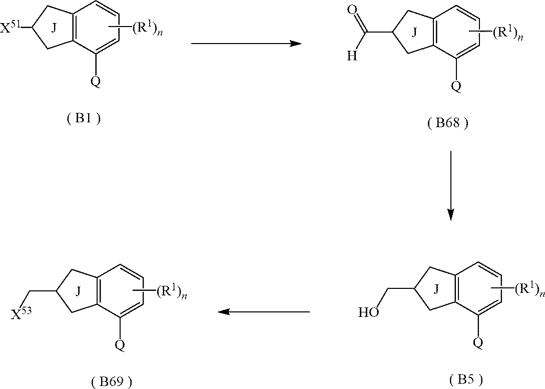

[wherein the symbols are the same as defined above.]

The Step (d-1) may be carried out according to the method described in Angew. Chem. Int. Ed., 2013, 52, 8611-8615 or the like.

The Step (d-2) may be carried out according to the method described in Chemistry-A European Journal, 2019, 25(15), 3950-3956 or the like.

The Step (d-3) may be carried out according to the method described in J. Org. Synth., 1974, 54, 63 or the like.

### Reference Production Method E

The Compound (B6) may be prepared by reacting the Compound (B2) with an oxidizing agent.

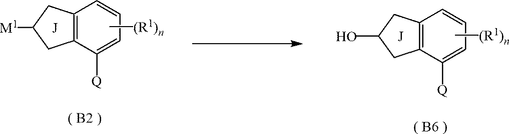

[wherein the symbols are the same as defined above.]

The reaction is usually carried out in a solvent. Examples of the solvent to be used in the reaction include hydrocarbons, ethers, halogenated hydrocarbons, amides, esters, nitriles, alcohols, water, and mixtures of two or more of them.

Examples of the oxidizing agent to be used in the reaction include m-chloroperoxybenzoic acid (hereinafter referred to as “mCPBA”) and hydrogen peroxide water.

When hydrogen peroxide water is used as the oxidizing agent, a base may be used as needed.

Examples of the base to be used in the reaction include alkali metal hydroxides.

When a base is used in the reaction, the base is usually used at a ratio of 0.1 to 5 mol relative to 1 mol of the Compound (B2).

In the reaction, the oxidizing agent is usually used at a ratio of 1 to 5 mol relative to 1 mol of the Compound (B2) .

The reaction temperature is usually within the range of -20 to 120° C. The reaction time is usually within the range of 0.1 to 48 hour(s).

When the reaction is completed, the reaction mixture may be subjected to a work-up such as adding water and a reducing agent such as sodium thiosulfate to the reaction mixture, extracting the resulting mixture with organic solvent(s), and drying and/or concentrating the resulting organic layer to isolate the Compound (B6).

### Reference Production Method F

A compound represented by formula (B71) (hereinafter referred to as “Compound (B71)”) may be prepared by reacting a compound represented by formula (B70) (hereinafter referred to as “Compound (B70)”) with bis(pinacolato)diboron in the presence of a base and a palladium catalyst.

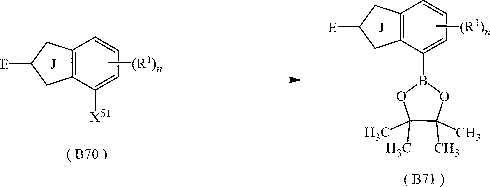

[wherein the symbols are the same as defined above.]

The reaction may be carried out according to the Reference production method a by using the Compound (B70) instead of the Compound (B1).

The Compound (B70) is known or may be prepared according to known method(s).

The Present compound is usually used by mixing it with inert carrier(s) such as solid carrier(s), liquid carrier(s), and gaseous carrier(s), surfactant(s), and the like, and as needed, adding thereto auxiliary agent(s) for formulation such as binder(s), dispersant(s), and stabilizer(s) to be formulated into an aqueous suspension formulation, an oily suspension formulation, an oil solution, an emulsifiable concentrate, an emulsion formulation, a microemulsion formulation, a microcapsule formulation, a wettable powder, a granular wettable powder, a dust formulation, a granule, a tablet, an aerosol formulation, a resin formulation, or the like. In addition to these formulations, the Present compound may be used by formulating it into a dosage form described in Manual on development and use of FAO and WHO Specifications for pesticides, FAO Plant Production and Protection Papers-271-276, prepared by the FAO/WHO Joint Meeting on Pesticide Specifications, 2016, ISSN: 0259-2517.

These formulations usually comprise 0.0001 to 99% by weight ratio of the Present compound.

Examples of the solid carrier include fine powders and granules of clays (for example, pyrophyllite clay and kaolin clay), talc, calcium carbonate, diatomaceous earth, zeolite, bentonite, acid white clay, attapulgite, white carbon, ammonium sulfate, vermiculite, perlite, pumice, silica sand, chemical fertilizers (for example, ammonium sulfate, ammonium phosphate, ammonium nitrate, urea, and ammonium chloride), and the others; as well as resins (for example, polypropylene, polyester, polyurethane, polyamide, and polyvinyl chloride).

Examples of the liquid carrier include water, alcohols (for example, ethanol, cyclohexanol, benzyl alcohol, propylene glycol, and polyethylene glycol), ketones (for example, acetone and cyclohexanone), aromatic hydrocarbons (for example, xylene, phenyl xylyl ethane, and methylnaphthalene), aliphatic hydrocarbons (for example, hexane and cyclohexane), esters (for example, ethyl acetate, methyl oleate, and propylene carbonate), nitriles (for example, acetonitrile), ethers (for example, ethylene glycol dimethyl ether), amides (for example, N,N-dimethylformamide and N,N-dimethyloctanamide), sulfoxides (for example, dimethylsulfoxide), lactams (for example, N-methylpyrrolidone and N-octylpyrrolidone), fatty acids (for example, oleic acid), and vegetable oils (for example, soybean oil) .

Examples of the gaseous carrier include fluorocarbon, butane gas, LPG (liquefied petroleum gas), dimethyl ether, nitrogen, and carbon dioxide.

Examples of the surfactant include nonionic surfactants (for example, polyoxyethylene alkyl ethers, polyoxyethylene alkyl aryl ethers, and polyethylene glycol fatty acid esters), and anionic surfactants (for example, alkyl sulfonates, alkyl aryl sulfonates, and alkyl sulfates).

Examples of the other auxiliary agent for formulation include binders, dispersants, colorants, and stabilizers, and the specific examples thereof include polysaccharides (for example, starch, gum arabic, cellulose derivatives, and alginic acid), lignin derivatives, water-soluble synthetic polymers (for example, polyvinyl alcohol, polyvinyl pyrrolidone, and polyacrylic acids), acidic isopropyl phosphate, and dibutylhydroxytoluene.

Examples of the method for applying the Present compound include a method for spraying it to soybean foliage, a method for treating it to seeds, and a method for applying it to soil for cultivating soybeans.

The amount of the Present compound to be applied may be varied depending on the climate condition, dosage form, application period, application method, application site, diseases to be controlled, crops to be protected, and the like. In case of spray to soybean foliage or application to soil for cultivating soybeans, the amount of the Present compound is usually within the range of 1 to 500 g, preferably 2 to 200 g, per 1000 m². In case of treatment to seeds, the amount of the Present compound to be applied is usually within the range of 0.001 to 100 g, preferably 0.01 to 50 g, per 1 Kg of seeds. An emulsifiable concentrate, a wettable powder, a suspension, and the like are usually diluted with water and then applied. In this case, the concentration of the Present compound after dilution is usually within the range of 0.0005 to 2% by weight, preferably 0.005 to 2% by weight. A dust formulation, a granule, and the like are usually applied as themselves without diluting them.

The above soybeans may be soybeans producible by natural crossing, soybeans producible by a mutation, F1 hybrid soybeans, and transgenic soybeans (also referred to as “genetically modified soybeans”). These soybeans generally have characteristics such as resistance to herbicides, accumulation of substances harmful to pests (also referred to as “resistance to pests”), sensitivity suppression against diseases (also referred to as “resistance to diseases”), increase in yield potential, improvement in resistance to biotic and abiotic stress factors, and quality modification of products (for example, increase and decrease in component contents, change in composition, and improvement in preservability or processability). Examples of technique for producing the above soybeans include conventional breeding techniques; gene-recombination techniques; genome breeding techniques; new breeding techniques; and genome-editing techniques.

Examples of soybeans having resistance to herbicides (soybeans resistant to herbicides) include soybeans resistant to auxin type herbicides such as 2,4-D and dicamba; soybeans resistant to glufosinate, soybeans resistant to glyphosate, soybeans resistant to isoxaflutole, soybeans resistant to 4-hydroxyphenylpyruvate dioxygenase inhibiting herbicides such as mesotrione; soybeans resistant to acetolactate synthase (ALS) inhibiting herbicides such as imidazolinone herbicides and sulfonylurea herbicides; and soybeans resistant to protoporphyrinogen oxidase inhibiting herbicides such as flumioxazin.

Soybeans that have acquired resistance to herbicides by gene-recombination techniques may be obtained by introducing a foreign gene (for example, gene of another organism such as a microorganism). For example, resistance to 2,4-D may be obtained by introducing a gene “aad-12” derived from Delftia acidovorans; resistance to dicamba may be obtained by introducing a gene “dmo” derived from Stenotrophomonas maltophilia strain DI-6; resistance to glufosinate may be obtained by introducing a gene “bar” derived from Streptomyces hygroscopicus or a gene “pat” derived from Streptomyces viridochromogenes; resistance to glyphosate may be obtained by introducing a gene “2mepsps” derived from Zea mays, a gene “CP4 epsps” derived from Agrobacterium tumefaciens strain CP4, or a gene “gat4601” derived from Bacillus licheniformis; resistance to isoxaflutole may be obtained by introducing a gene “hppdPF W336” derived from Pseudomonas fluorescens strain A32; resistance to mesotrione may be obtained by introducing a gene “avhppd-03” derived from Oat (Avena sativa); resistance to imidazolinone herbicides may be obtained by introducing a gene “csr1-2” derived from Arabidopsis thaliana; and resistance to sulfonylurea herbicides may be obtained by introducing a gene “gm-hra” derived from Glycine max.

Examples of soybeans that have acquired resistance to herbicides by a conventional breeding technique or a genome breeding technique include a soybean “STS (registered trademark) soybean” having resistance to sulfonylurea ALS inhibiting herbicides such as thifensulfuron-methyl.

Examples of soybeans that have acquired resistance to herbicides by a new breeding technique include a soybean in which a Roundup Ready (registered trademark) soybean having resistance to glyphosate is used as a rootstock, thereby resistance to glyphosate have been added to a nontransgenic soybeans graft (see Weed Technology, 2013, 27, 412.).

Examples of soybeans having resistance to pests include soybeans having resistance to Lepidoptera pests (for example, Pseudoplusia includens, Helicoverpa zea, and Spodoptera frugiperda), soybeans having resistance to Hemiptera pests (for example, Aphis glycines), and soybeans having resistance to Nematoda (for example, Heterodera glycines and Meloidogyne incognita).

Soybeans that have acquired resistance to pests by a gene-recombination technique may be obtained by introducing a foreign gene (for example, a gene encoding an insecticidal protein δ-endotoxin derived from Bacillus thuringiensis). For example, resistance to Lepidoptera pests may be obtained by introducing a gene “cry1Ac” derived from Bacillus thuringiensis subsp. Kurstaki strain HD73, a gene “cry1F” derived from Bacillus thuringiensis var. aizawai, a gene “cry1A.105” derived from Bacillus thuringiensis subsp. kumamotoensis, or a gene “cry2Ab2” derived from Bacillus thuringiensis subsp. kumamotoensis.

Examples of soybeans that have acquired resistance to pests by a conventional breeding technique or a genome breeding technique include a soybean having a gene showing resistance to soybean aphid, i.e., a “Rag1 (Resistance to Aphis glycines 1)” or a “Rag2 (Resistance to Aphis glycines 2)” gene, thereby showing resistance to soybean aphid (Aphis glycines) (see J. Econ. Entomol., 2015, 108, 326.); a soybean showing resistance to soybean cyst nematode (Heterodera glycines) (see Phytopathology, 2016, 106, 1444.); and a soybean “Fukuminori” showing resistance to cotton worm (Spodoptera litura).

Examples of soybeans that have acquired resistance to diseases include cultivars that have acquired resistance to soybean rust by a conventional breeding techniques or a gene-recombination technique. Examples of frequently used resistant gene include, but are not limited to, Rpp1, Rpp2, Rpp3, Rpp4, Rpp5, and Rpp6. Any one of these genes may be inserted into a soybean, or a combination of two or more of them may be inserted into a soybean. These genes are described in the following academic literatures and the like. Crop Science, 2007, 47, 837.; Theoretical and Applied Genetics, 2008, 117, 57.; Theoretical and Applied Genetics, 117, 545.; Crop Science, 2009, 49, 783.; Theoretical and Applied Genetics, 2009, 119, 271.; Theoretical and Applied Genetics, 2010, 121, 1023.; Theoretical and Applied Genetics, 2012, 125, 133.

Examples of soybeans that have acquired resistance to diseases by a genome-editing technique include a soybean wherein a RXLR effector gene (Avr4/6) is disrupted by using CRISPR-Cas9, thereby shows resistance to soybean Phytophthora root rot caused by Phytophthora sojae (see Mol. Plant. Pathol., 2016, 17, 127.).

Also, there are soybeans that have acquired resistance to soybean diseases other than soybean rust (for example, frogeye leaf spot, target spot, Phytophthora root rot, and sudden death syndrome).

Examples of soybeans in which the product quality is modified by a gene-recombination technique include a soybean “Plenish (trademark)” or “Treus (trademark)” in which a partial gene “gm-fad2-1” of ω-6 desaturase that is a desaturase of fatty acid derived from Glycine max is introduced, thereby the expression of said gene is suppressed, and the oleic acid content is increased; a soybean “Vistive Gold (trademark)” in which a gene that produces a double-stranded RNA of an acyl-acyl carrier protein thioesterase gene “fatb1-A” derived from Glycine max and a gene that produces a double-stranded RNA of a δ-12 desaturase gene “fad2-1A” derived from Glycine max are introduced, thereby the saturated fatty acid content is decreased; a soybean in which a δ-6 desaturase gene “Pj.D6D” derived from Primula juliae and a δ-12 desaturase gene “Nc.Fad3” derived from Neurospora crassa are introduced, thereby a ω3 fatty acid, stearidonic acid is produced; a soybean in which the oil content is modified; a soybean in which the allergen content is decreased (see U.S. Pat. No. 6864362); a soybean in which the lysine content is increased (see Bio/Technology, 1995, 13, 577.); a soybean in which the composition of methionine, leucine, isoleucine, and valine is modified; a soybean in which the sulfur amino acid content is increased (see WO 1997/041239 pamphlet); a soybean in which the phenolic compound content is modified (see U.S. Pat. Application Publication No. 2008/235829); and a soybean in which the vitamin E content is increased (see WO 2004/058934 pamphlet).

Examples of soybeans in which the product quality is modified by a genome breeding technique include a soybean “Yumeminori” in which the allergen content is decreased.

Examples of soybeans in which a character pertaining to plant growth or yield is modified include a soybean in which a gene “bbx32” encoding a transcription factor regulating the circadian derived from Arabidopsis thaliana is introduced, thereby the plant growth is enhanced, and as a result, a high yield is expected.

Examples of soybeans having other characteristics include a soybean in which the phosphorus uptake is improved; a soybean that has acquired the fertility characters; a soybean that has acquired resistance to drought; a soybean that has acquired resistance to low temperature; a soybean that has acquired resistance to high salinity; a soybean in which the iron chlorosis is improved; and a soybean in which the chloride sensitivity is modified.

The above soybeans also encompass soybeans that have acquired two or more of the above-mentioned resistance to herbicides, resistance to pests, resistance to diseases, resistance to abiotic stresses, characters pertaining to growth or yield, characters pertaining to nutrient uptake, characters pertaining to product quality, fertility characters, and the like. Examples thereof include resistance to glyphosate; resistance to glufosinate; resistance to frogeye leaf spot, sudden death syndrome, southern stem canker, Phytophthora root rot, southern root-knot nematode, Sclerotinia white mold, brown stem rot, and soybean cyst nematode; improvement of iron chlorosis, and a soybean “Credenz (registered trademark) soybean” in which the chloride sensitivity is modified.

Hereinafter, commercially available or developed soybeans are recited. The following soybeans are represented by [Event Name, Event code, Tread name]. Also, the symbol of “NA” means no information or unavailable information. Many of these soybeans are listed in a registration database (GM APPROVAL DATABASE) in a website (http://www.isaaa.org/) of the INTERNATINAL SERVICE for the ACQUISITION of AGRI-BIOTECH APPLICATIONS (ISAAA).

05 (G94-1, G94-19, G168), DD-026005-3, NA], [A2704-12, ACS-GM005-3, Liberty Link (trademark) soybean], [A2704-21, ACS-GM004-2, Liberty Link (trademark) soybean], [A5547-127, ACS-GM006-4, Liberty Link (trademark) soybean], [A5547-35, ACS-GM008-6, Liberty Link (trademark) soybean], [CV127, BPS-CV127-9, Cultivance], [DAS44406-6, DAS-44406-6, NA], [DAS68416-4, DAS-68416-4, Enlist (trademark) Soybean], [DAS68416-4xMON89788, DAS-68416-4xMON-89788-1, NA], [DAS81419, DAS-81419-2, NA], [DAS81419xDAS44406-6, DAS-81419-2xDAS-44406-6, NA], [DP305423, DP-305423-1, Treus (trademark) or Plenish (trademark)], [DP305423xGTS40-3-2, DP-305423-1xMON-04032-6, NA], [DP356043, DP-356043-5, Optimum GAT (trademark)], [FG72(FG072-2,FG072-3), MST-FG072-3, NA], [FG72xA5547-127, MST-FG072-3xACS-GM006-4, NA], [GTS40-3-2(40-3-2), MON-04032-6, Roundup Ready (trademark) soybean], [GU262, ACS-GM003-1, Liberty Link (trademark) soybean], [IND-00410-5, IND-00410-5, Verdeca HB4 Soybean], [MON87701, MON-87701-2, NA], [MON87701xMON89788, MON-87701-2xMON-89788-1, Intacta (trademark) Roundup Ready (trademark) 2 Pro], [MON87705, MON-87705-6, Vistive Gold (trademark)], [MON87705xMON87708, MON-87705-6xMON-87708-9, NA], [MON87705xMON87708xMON89788, MON-87705-6xMON-87708-9xMON-89788-1, NA], [MON87705xMON89788, MON-87705-6xMON-89788-1, NA], [MON87708, MON-87708-9, Genuity (registered trademark) Roundup Ready (trademark) 2 Xtend (trademark)], [MON87708xMON89788, MON-87708-9xMON-89788-1, Roundup Ready 2 Xtend (registered trademark) ], [MON87712, MON-87712-4, NA], [MON87751, MON-87751-7, NA], [MON87751xMON87701xMON87708xMON89788, MON-87751-7xMON-87701-2xMON87708xMON89788, NA], [MON87769, MON87769-7, NA], [, MON87769xMON89788, MON-87769-7xMON-89788-1, NA], [MON89788, MON-89788-1, Genuity (registered trademark) Roundup Ready 2 Yield (trademark)], [SYHT0H2, SYN-000H2-5, Herbicide-tolerant Soybean line], [W62, ACS-GM002-9, Liberty Link (trademark) soybean], [W98, ACS-GM001-8, Liberty Link (trademark) soybean], [OT96-15, OT96-15, NA], [NA, NA, STS (registered trademark) soybean], [NA, NA, Credenz (registered trademark) soybean], [NA, NA, Enlist E3 (trademark)], [NA, NA, Enlist (trademark) Roundup Ready 2 Yield (registered trademark)], [NA, NA, Fukuminori], [NA, NA, Yumeminori], [DP305423 x MOV87708, DP-305423-1 x MON-87708-9, NA], [DP305423 x MOV87708 x MON89788, DP-305423-1 x MON-87708-9 x MON-89788-1, NA], [DP305423 x MON89788, DP-305423-1 x MON-89788-1, NA]

Applying the Present compound to a soybean achieves effects for promoting the plant growth such as the increase in the rate of seedling establishment, increase in the number of healthy leaves, increase in the height of the plant, increase in the weight of the plant, increase in the leaf area, increase in the number or weight of seeds, increase in the number of occasion of flower setting or fruit setting, and promotion in the growth of a root.

Also, applying the Present compound to a soybean achieves the improvement in resistance to abiotic stresses such as temperature stresses (for example, high temperature stress and low temperature stress), water stresses (for example, drought stress and excess water stress), or salt stresses.

### EXAMPLES

Hereinafter, the present invention is illustrated more in detail by Preparation Examples, Formulation Examples, and Test Examples, but the present invention is not limited to these Examples only.

In the present description, Et represents an ethyl group, Pr represents a propyl group, i-Pr represents an isopropyl group, c-Pr represents a cyclopropyl group, Bu represents a butyl group, and Ph represents a phenyl group.

Preparation Examples of the Present compound are shown below.

When a physical property of a compound is measured by liquid chromatography / mass spectrometry (hereinafter referred to as “LCMS”), the measured molecular ion value [M+H]⁺ or [M-H]⁻, and retention time (hereinafter referred to as “RT”) are described. The conditions of liquid chromatography (hereinafter referred to as “LC”) and mass spectrometry (hereinafter referred to as “MS”) are as follows.

### LC Conditions

Column: L-column2 ODS, inner diameter: 4.6 mm, length: 30 mm, particle size: 3 µm (Chemicals Evaluation and Research Institute, Japan)
UV measurement wavelength: 254 nm
Mobile phase: Solution A: 0.1% formic acid in water,
Solution B: 0.1% formic acid in acetonitrile
Flow rate: 2.0 mL/min
Pump: two LC-20AD (manufactured by Shimadzu Corporation) (high pressure gradient)
Gradient condition: sending a solution with the concentration gradient described in Table LC1.

TABLE LC1

| Time (min) | Solution A (%) | Solution B (%) |
|---|---|---|
| 0.01 | 90 | 10 |
| 2.00 | 0 | 100 |
| 4.00 | 0 | 100 |
| 4.01 | 90 | 10 |

### MS Conditions

Detector: LCMS-2020 (manufactured by Shimadzu Corporation) Ionization method: DUIS

### Reference Preparation Example 1

A mixture of benzoyl chloride (0.28 g), 2-amino-3-bromophenol (0.28 g), and toluene (4 mL) was stirred at 80° C. for 3 hours. To the resulting mixture was added water, and the resulting mixture was extracted with ethyl acetate. The resulting organic layer was sequentially washed with water and saturated brine, dried over anhydrous sodium sulfate, and concentrated under reduced pressure.

To the resulting crude product were added p-toluenesulfonic acid monohydrate (0.19 g) and toluene (5 mL), and the resulting mixture was stirred under reflux for 4 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography to give the Intermediate compound 1 represented by the following formula (0.60 g).

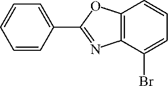 [SMILES: Brc1cccc2oc(-c3ccccc3)nc12]

Intermediate compound 1: ¹H-NMR (CDCl₃) δ: 8.31 (2H, dd), 7.58-7.50 (5H, m), 7.23 (1H, d).

### Reference Preparation Example 1-1

The compound prepared according to the Reference Preparation Example 1 and a physical property thereof are shown below.

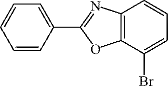 [SMILES: Brc1cccc2nc(-c3ccccc3)oc12]

Intermediate compound 5: ¹H-NMR (CDCl₃) δ: 8.30 (2H, dd), 7.71 (1H, d), 7.58-7.54 (3H, m), 7.50 (1H, d), 7.26-7.23 (1H, m).

### Reference Preparation Example 2

A mixture of the Intermediate compound 1 (0.50 g), bis(pinacolato)diboron (0.51 g), [1,1′-bis(diphenylphosphino)ferrocene]palladium(II) dichloride (0.04 g), potassium acetate (0.53 g), and DMSO (6 mL) was stirred at 100° C. for 7 hours. To the resulting mixture was added water, and the resulting mixture was extracted with ethyl acetate. The resulting organic layer was sequentially washed with water and saturated brine, dried over anhydrous magnesium sulfate, and concentrated under reduced pressure to give the Intermediate compound 2 represented by the following formula (0.60 g).

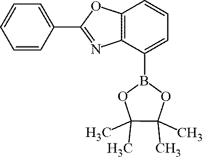 [SMILES: CC1(C)OB(c2cccc3oc(-c4ccccc4)nc23)OC1(C)C]

Intermediate compound 2: ¹H-NMR (CDCl₃) δ: 8.34-8.31 (2H, m), 7.80 (1H, dd), 7.67 (1H, dd), 7.55-7.50 (3H, m), 7.35 (1H, dd), 1.45 (12H, s).

### Reference Preparation Example 2-1

The compound prepared according to the Reference Preparation Example 2 and a physical property thereof are shown below.

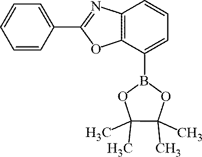 [SMILES: CC1(C)OB(c2cccc3nc(-c4ccccc4)oc23)OC1(C)C]

Intermediate compound 6: ¹H-NMR (CDCl₃) δ: 8.31-8.29 (2H, m), 7.87 (1H, dd), 7.76 (1H, dd), 7.56-7.52 (3H, m), 7.36 (1H, t), 1.44 (12H, s).

### Reference Preparation Example 3

A mixture of benzoyl chloride (2.81 g), 2,6-dibromoaniline (5.02 g), toluene (4 mL), and pyridine (4 mL) was stirred at 90° C. for 6 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting solid crude product was washed with MTBE to give the Intermediate compound 3 represented by the following formula (5.85 g).

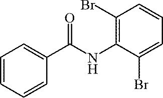 [SMILES: O=C(Nc1c(Br)cccc1Br)c1ccccc1]

Intermediate compound 3: ¹H-NMR (CDCl₃) δ: 7.98 (2H, d), 7.65-7.58 (4H, m), 7.52 (2H, t), 7.08 (1H, t).

### Reference Preparation Example 4

A mixture of the Intermediate compound 3 (1.78 g), Lawesson’s reagent (1.22 g), and chlorobenzene (30 mL) was stirred under reflux for 1 day. To the resulting mixture was added a saturated aqueous solution of sodium hydrogen carbonate, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography to give the Intermediate compound 4 represented by the following formula (0.47 g).

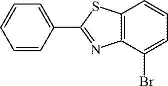 [SMILES: Brc1cccc2sc(-c3ccccc3)nc12]

Intermediate compound 4: ¹H-NMR (CDCl₃) δ: 8.16-8.12 (2H, m), 7.84 (1H, d), 7.69 (1H, d), 7.52-7.49 (3H, m), 7.23 (1H, dd).

### Reference Preparation Example 5

To a mixture of 8-aminonaphthalen-2-ol (10 g), triethylamine (12 mL), and THF (200 mL) was added N-phenylbis(trifluoromethanesulfonimide) (25 g) under ice-cooling, and the resulting mixture was stirred at room temperature for 1 day. The resulting mixture was concentrated under reduced pressure. The resulting residue was subjected to silica gel column chromatography to give the Intermediate compound 7 represented by the following formula (18.5 g).

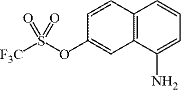 [SMILES: Nc1cccc2ccc(OS(=O)(=O)C(F)(F)F)cc12]

Intermediate compound 7: ¹H-NMR (CDCl₃) δ: 7.87 (1H, d), 7.72 (1H, d), 7.41-7.30 (3H, m), 6.88 (1H, dd), 4.13 (2H, br s).

### Reference Preparation Example 6

A mixture of the Intermediate compound 7 (5.82 g), phenylboronic acid (3.66 g), {1,1′-bis(diphenylphosphino)ferrocene}dichloropalladium(II) (1.46 g), tripotassium phosphate (12.3 g), and DME (40 mL) was stirred under reflux for 1 day. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography to give the Intermediate compound 8 represented by the following formula (3.89 g).

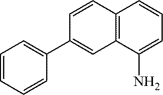 [SMILES: Nc1cccc2ccc(-c3ccccc3)cc12]

Intermediate compound 8: ¹H-NMR (CDCl₃) δ: 7.99 (1H, s), 7.88 (1H, d), 7.72 (3H, d), 7.49 (2H, t), 7.40-7.26 (3H, m), 6.82 (1H, d), 4.21 (2H, br s).

### Reference Preparation Example 7

To a mixture of the Intermediate compound 8 (0.44 g) and methanol (4 mL) was added 3 M hydrochloric acid (2 mL) under ice-cooling, and the resulting mixture was stirred at 0° C. for 5 minutes. To the resulting mixture was added a mixture of sodium nitrite (0.16 g) and water (1 mL) under ice-cooling, and the resulting mixture was stirred at 0° C. for 2 hours. To the resulting mixture was added bis(pinacolato)diboron (1.52 g) under ice-cooling, and the resulting mixture was stirred at room temperature for 2 hours. To the resulting mixture was added water, and the resulting mixture was extracted with ethyl acetate. The resulting organic layer was sequentially washed with water and saturated brine, dried over anhydrous sodium sulfate, and concentrated under reduced pressure to give a crude product of the Intermediate compound 9 represented by the following formula (0.70 g).

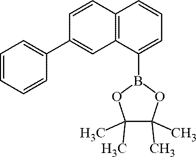 [SMILES: CC1(C)OB(c2cccc3ccc(-c4ccccc4)cc23)OC1(C)C]

Intermediate compound 9: ¹H-NMR (CDCl₃) δ: 9.05 (1H, s), 8.09 (1H, d), 7.95 (1H, d), 7.90 (1H, d), 7.79-7.74 (3H, m), 7.53-7.45 (3H, m), 7.38 (1H, t), 1.43 (12H, s).

### Preparation Example 1

A mixture of the Intermediate compound 2 (0.60 g), tris(dibenzylideneacetone)dipalladium(0) (0.18 g), methyl 2-iodo-3-methoxyacrylate (0.44 g), SPhos (0.16 g), tripotassium phosphate (1.54 g), and toluene (15 mL) was stirred at 110° C. for 6 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (ethyl acetate : hexane = 1 : 1) to give the Present compound 2 represented by the following formula (0.25 g).

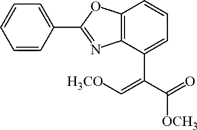 [SMILES: CO/C=C(/C(=O)OC)c1cccc2oc(-c3ccccc3)nc12]

Present compound 2: ¹H-NMR (CDCl₃) δ: 8.27-8.22 (2H, m), 7.72 (1H, s), 7.54-7.47 (4H, m), 7.35 (1H, t), 7.29-7.26 (1H, m), 3.88 (3H, s), 3.74 (3H, s).

### Preparation Example 2

To a mixture of the Present compound 1 (0.15 g) and THF (2 mL) was added methylamine (9.8 M solution in methanol) (1 mL), and the resulting mixture was stirred at room temperature overnight. To the resulting mixture was added water, and the resulting mixture was extracted with ethyl acetate. The resulting organic layer was dried over anhydrous sodium sulfate, and concentrated under reduced pressure. The resulting residue was subjected to silica gel column chromatography (ethyl acetate : hexane = 1 : 1) to give the Present compound 3 represented by the following formula (0.04 g) and the Present compound 4 represented by the following formula (0.12 g).

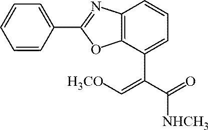 [SMILES: CNC(=O)/C(=C/OC)c1cccc2nc(-c3ccccc3)oc12]

Present compound 3: ¹H-NMR (CDCl₃) δ: 8.42 (1H, br s), 8.28-8.23 (2H, m), 7.54-7.48 (3H, m), 7.44-7.26 (4H, m), 3.72 (3H, s), 3.13 (3H, d).

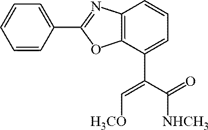 [SMILES: CNC(=O)/C(=C\OC)c1cccc2nc(-c3ccccc3)oc12]

Present compound 4: ¹H-NMR (CDCl₃) δ: 8.24-8.21 (2H, m), 7.76 (1H, d), 7.70 (1H, d), 7.47-7.32 (5H, m), 4.47 (1H, br s), 3.70 (3H, s), 2.99 (3H, d).

### Preparation Example 3

A mixture of the Intermediate compound 4 (1.0 g), bis(pinacolato)diboron (0.96 g), [1,1′-bis(diphenylphosphino)ferrocene]palladium(II) dichloride (0.08 g), potassium acetate (1.04 g), and DMSO (10 mL) was stirred at 90° C. for 8 hours. To the resulting mixture was added water, and the resulting mixture was extracted with ethyl acetate. The resulting organic layer was sequentially washed with water and saturated brine, dried over anhydrous magnesium sulfate, and concentrated under reduced pressure. A mixture of the resulting crude product, tris(dibenzylideneacetone)dipalladium(0) (0.31 g), methyl 2-iodo-3-methoxyacrylate (0.84 g), SPhos (0.29 g), tripotassium phosphate (2.93 g), and toluene (20 mL) was stirred at 110° C. for 7 hours. To the resulting mixture was added water, and the resulting mixture was extracted with ethyl acetate. The resulting organic layer was sequentially washed with water and saturated brine, dried over anhydrous sodium sulfate, and concentrated under reduced pressure. The resulting residue was subjected to silica gel column chromatography (ethyl acetate : hexane = 1 : 1) to give the Present compound 6 represented by the following formula (0.31 g).

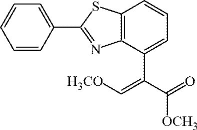 [SMILES: CO/C=C(/C(=O)OC)c1cccc2sc(-c3ccccc3)nc12]

Present compound 6: ¹H-NMR (CDCl₃) δ: 8.06 (2H, m), 7.85 (1H, dd), 7.68 (1H, s), 7.46 (3H, dd), 7.44-7.37 (2H, m), 3.86 (3H, s), 3.71 (3H, s).

### Preparation Example 4

The compounds prepared according to the methods described in the Production methods, Preparation Examples, JPH 8-27133 A, or the like, and physical properties thereof are shown below.

A compound represented by formula (I):

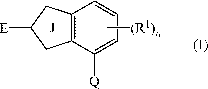

wherein n represents 0, and the combination of E, J, and Q represents any one combination indicated in Table T1.

The term of “Comp” described in Table T1 means the Present compound number.

E represents any one of substituent numbers 1 to 195 described in Table V1 to Table V7. The term of “sub number” described in Table V1 to Table V7 means the substituent number.

For example, the compound wherein Comp (Present compound number) is 4 described in Table T1, namely the Present compound 4 means a compound wherein E represents the group of sub number (substituent number) 25 described in Table V1, J represents the group represented by J2-4, and Q represents the group represented by Q1-5. Specifically, the Present compound 4 is a compound of the following structure.

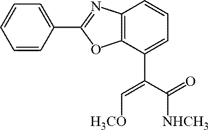 [SMILES: CNC(=O)/C(=C\OC)c1cccc2nc(-c3ccccc3)oc12]

TABLE V1

| sub number | E | sub number | E | sub number | E |
|---|---|---|---|---|---|
| 1 | 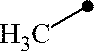 | 11 | 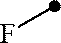 | 21 | 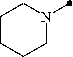 |
| 2 | 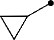 | 12 | 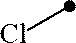 | 22 | 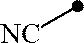 |
| 3 | 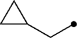 | 13 | 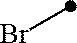 | 23 | 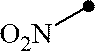 |
| 4 | 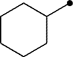 | 14 | 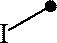 | 24 | 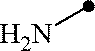 |
| 5 | 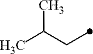 | 15 | 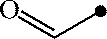 | 25 | 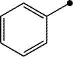 |
| 6 | 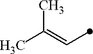 | 16 | 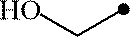 | 26 | 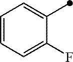 |
| 7 | 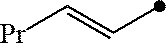 | 17 | 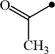 | 27 | 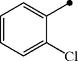 |
| 8 | 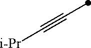 | 18 | 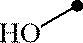 | 28 | 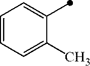 |
| 9 | 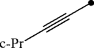 | 19 | 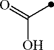 | 29 | 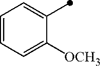 |
| 10 | 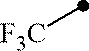 | 20 | 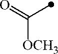 | 30 | 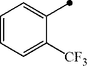 |

TABLE V2

| sub number | E | sub number | E | sub number | E |
|---|---|---|---|---|---|
| 31 | 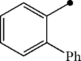 | 41 | 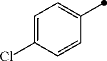 | 51 | 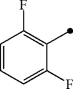 |
| 32 | 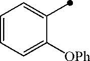 | 42 | 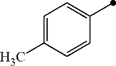 | 52 | 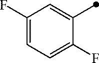 |
| 33 | 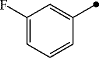 | 43 | 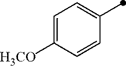 | 53 | 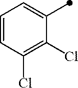 |
| 34 | 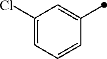 | 44 | 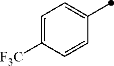 | 54 | 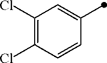 |
| 35 | 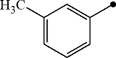 | 45 | 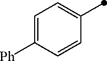 | 55 | 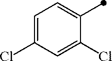 |
| 36 | 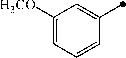 | 46 | 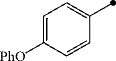 | 56 | 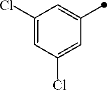 |
| 37 | 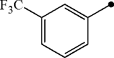 | 47 | 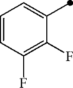 | 57 | 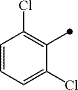 |
| 38 | 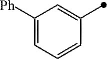 | 48 | 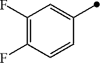 | 58 | 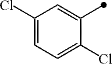 |
| 39 | 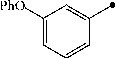 | 49 | 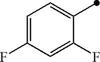 | 59 | 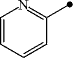 |
| 40 | 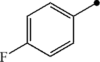 | 50 | 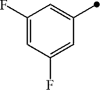 | 60 | 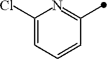 |

TABLE V3

| sub number | E | sub number | E | sub number | E |
|---|---|---|---|---|---|
| 61 | 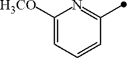 | 71 | 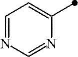 | 81 | 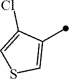 |
| 62 | 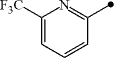 | 72 | 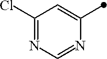 | 82 | 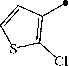 |
| 63 | 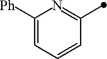 | 73 | 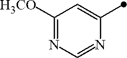 | 83 | 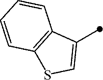 |
| 64 | 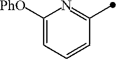 | 74 | 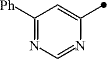 | 84 | 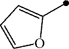 |
| 65 | 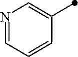 | 75 | 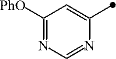 | 85 | 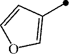 |
| 66 | 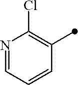 | 76 | 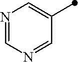 | 86 | 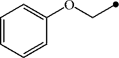 |
| 67 | 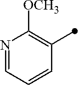 | 77 | 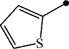 | 87 | 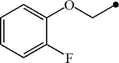 |
| 68 | 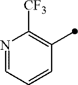 | 78 | 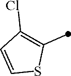 | 88 | 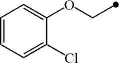 |
| 69 | 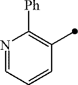 | 79 | 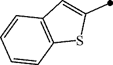 | 89 | 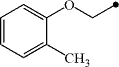 |
| 70 | 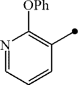 | 80 | 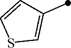 | 90 | 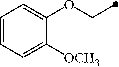 |

TABLE V4

| sub number | E | sub number | E | sub number | E |
|---|---|---|---|---|---|
| 91 | 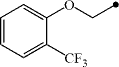 | 101 | 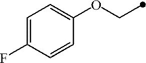 | 111 | 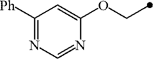 |
| 92 | 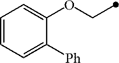 | 102 | 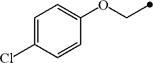 | 112 | 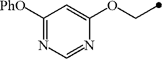 |
| 93 | 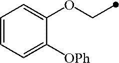 | 103 | 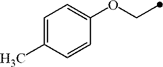 | 113 | 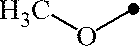 |
| 94 | 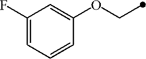 | 104 | 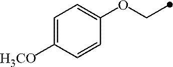 | 114 | 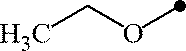 |
| 95 | 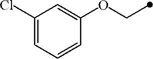 | 105 | 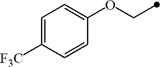 | 115 | 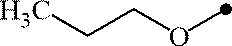 |
| 96 | 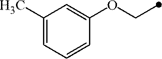 | 106 | 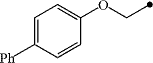 | 116 | 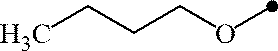 |
| 97 | 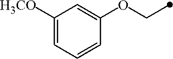 | 107 | 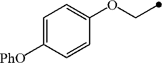 | 117 | 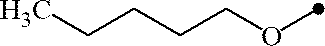 |
| 98 | 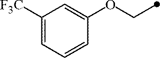 | 108 | 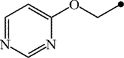 | 118 | 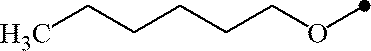 |
| 99 | 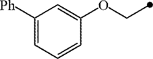 | 109 | 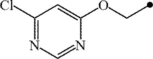 | 119 | 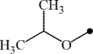 |
| 100 | 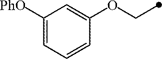 | 110 | 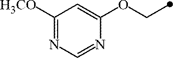 | 120 | 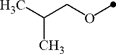 |

TABLE V5

| sub number | E | sub number | E | sub number | E |
|---|---|---|---|---|---|
| 121 | 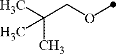 | 131 | 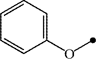 | 141 | 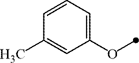 |
| 122 | 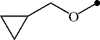 | 132 | 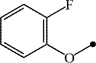 | 142 | 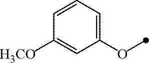 |
| 123 | 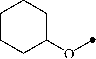 | 133 | 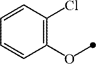 | 143 | 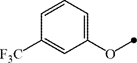 |
| 124 | 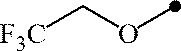 | 134 | 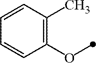 | 144 | 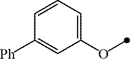 |
| 125 | 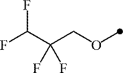 | 135 | 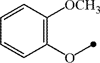 | 145 | 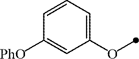 |
| 126 | 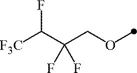 | 136 | 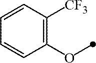 | 146 | 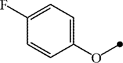 |
| 127 | 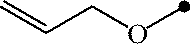 | 137 | 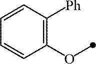 | 147 | 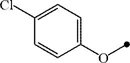 |
| 128 | 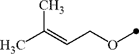 | 138 | 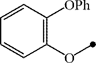 | 148 | 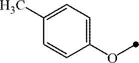 |
| 129 | 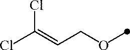 | 139 | 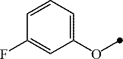 | 149 | 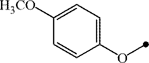 |
| 130 | 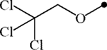 | 140 | 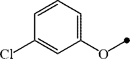 | 150 | 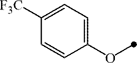 |

TABLE V6

| sub number | E | sub number | E | sub number | E |
|---|---|---|---|---|---|
| 151 | 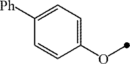 | 161 | 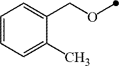 | 171 | 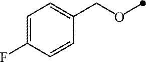 |
| 152 | 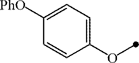 | 162 | 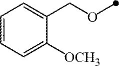 | 172 | 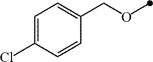 |
| 153 | 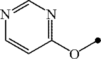 | 163 | 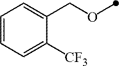 | 173 | 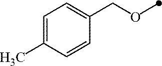 |
| 154 | 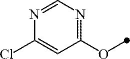 | 164 | 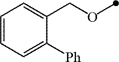 | 174 | 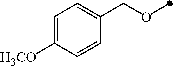 |
| 155 | 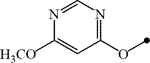 | 165 | 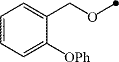 | 175 | 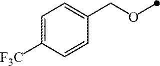 |
| 156 | 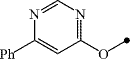 | 166 | 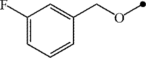 | 176 | 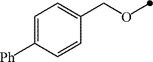 |
| 157 | 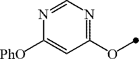 | 167 | 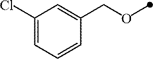 | 177 | 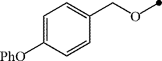 |
| 158 | 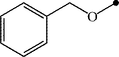 | 168 | 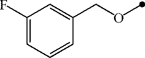 | 178 | 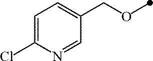 |
| 159 | 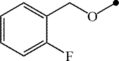 | 169 | 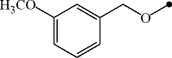 | 179 | 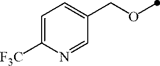 |
| 160 | 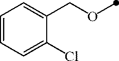 | 170 | 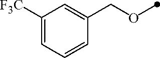 | 180 | 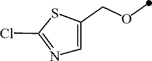 |

TABLE V7

| sub number | E | sub number | E |
|---|---|---|---|
| 181 | 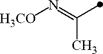 | 191 | 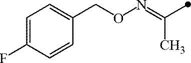 |
| 182 | 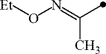 | 192 | 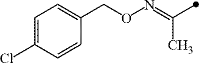 |
| 183 | 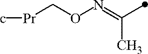 | 193 | 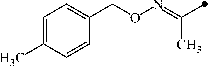 |
| 184 | 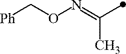 | 194 | 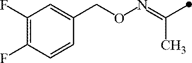 |
| 185 | 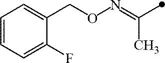 | 195 | 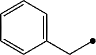 |
| 186 | 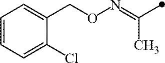 | | |
| 187 | 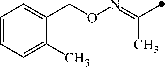 | | |
| 188 | 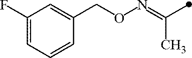 | | |
| 189 | 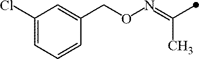 | | |
| 190 | 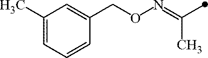 | | |

TABLE T1

| Comp | E | J | Q |
|---|---|---|---|
| 1 | 25 | J2-4 | Q1-1 |
| 2 | 25 | J1-4 | Q1-1 |
| 3 | 25 | J2-4 | Q1-4 |
| 4 | 25 | J2-4 | Q1-5 |
| 5 | 25 | J1-4 | Q1-4 |
| 6 | 25 | J1-5 | Q1-1 |
| 7 | 25 | J1-5 | Q1-4 |
| 8 | 25 | J1-7 | Q1-1 |
| 9 | 2 | J1-4 | Q1-1 |
| 10 | 2 | J2-4 | Q1-1 |
| 11 | 3 | J1-4 | Q1-1 |
| 12 | 103 | J2-7 | Q1-3 |
| 13 | 25 | J1-4 | Q1-2 |
| 14 | 25 | J1-4 | Q1-3 |
| 15 | 25 | J1-4 | Q1-6 |
| 16 | 41 | J1-4 | Q1-2 |
| 17 | 34 | J1-4 | Q1-2 |
| 18 | 34 | J1-4 | Q1-3 |
| 19 | 27 | J1-4 | Q1-2 |
| 20 | 27 | J1-4 | Q1-3 |
| 21 | 55 | J1-4 | Q1-2 |
| 22 | 77 | J1-4 | Q1-2 |
| 23 | 84 | J1-4 | Q1-2 |
| 24 | 84 | J1-4 | Q1-6 |
| 25 | 25 | J2-4 | Q1-2 |
| 26 | 25 | J2-4 | Q1-6 |
| 27 | 78 | J1-4 | Q1-2 |
| 28 | 78 | J1-4 | Q1-6 |
| 29 | 195 | J1-4 | Q2-1 |
| 30 | 195 | J1-4 | Q2-2 |
| 31 | 1 | J1-4 | Q2-1 |
| 32 | 25 | J1-4 | Q2-1 |
| 33 | 1 | J1-4 | Q2-2 |
| 34 | 25 | J1-4 | Q2-2 |
| 35 | 86 | J1-4 | Q2-1 |

Present compound 1: ¹H-NMR (CDCl₃) δ: 8.24-8.21 (2H, m), 7.75 (1H, s), 7.72 (1H, dd), 7.55-7.49 (3H, m), 7.36 (1H, t), 7.29 (1H, dd), 3.91 (3H, s), 3.76 (3H, s).

Present compound 5: ¹H-NMR (CDCl₃) δ: 8.42 (1H, br s), 8.27-8.24 (2H, m), 7.53-7.48 (3H, m), 7.43-7.29 (4H, m), 3.72 (3H, s), 3.13 (3H, d).

Present compound 7: ¹H-NMR (CDCl₃) δ: 8.31 (1H, br s), 8.08-8.04 (2H, m), 7.72 (1H, dd), 7.49-7.45 (3H, m), 7.39-7.23 (3H, m), 3.68 (3H, s), 3.11 (3H, d).

Present compound 8: ¹H-NMR (CDCl₃) δ: 7.94-7.91 (2H, m), 7.85 (1H, d), 7.81 (1H, s), 7.73 (1H, dd), 7.66 (2H, dd), 7.51-7.45 (3H, m), 7.39-7.35 (2H, m), 3.81 (3H, s), 3.68 (3H, s).
Present compound 9: LCMS: 274 [M+H]⁺, RT = 1.69 min
Present compound 10: LCMS: 274 [M+H]⁺, RT = 1.65 min
Present compound 11: LCMS: 288 [M+H]⁺, RT = 1.80 min
Present compound 12: LCMS: 368 [M+H]⁺, RT = 2.31 min
Present compound 13: LCMS: 311 [M+H]⁺, RT = 2.01 min
Present compound 14: LCMS: 311 [M+H]⁺, RT = 1.80 min
Present compound 15: LCMS: 310 [M+H]⁺, RT = 1.75 min
Present compound 16: LCMS: 345 [M+H]⁺, RT = 2.16 min
Present compound 17: LCMS: 345 [M+H]⁺, RT = 2.17 min
Present compound 18: LCMS: 345 [M+H]⁺, RT = 1.96 min
Present compound 19: LCMS: 345 [M+H]⁺, RT = 2.06 min
Present compound 20: LCMS: 345 [M+H]⁺, RT = 1.85 min
Present compound 21: LCMS: 379 [M+H]⁺, RT = 2.24 min
Present compound 22: LCMS: 317 [M+H]⁺, RT = 1.93 min
Present compound 23: LCMS: 301 [M+H]⁺, RT = 1.78 min
Present compound 24: LCMS: 300 [M+H]⁺, RT = 1.53 min
Present compound 25: LCMS: 311 [M+H]⁺, RT = 1.95 min
Present compound 26: LCMS: 310 [M+H]⁺, RT = 1.72 min
Present compound 27: LCMS: 351 [M+H]⁺, RT = 2.04 min
Present compound 28: LCMS: 350 [M+H]⁺, RT = 1.80 min
Present compound 29: LCMS: 341 [M+H]⁺, RT = 1.80 min
Present compound 30: LCMS: 337 [M+H]⁺, RT = 1.65 min
Present compound 31: LCMS: 265 [M+H]⁺, RT = 1.36 min
Present compound 32: LCMS: 327 [M+H]⁺, RT = 1.86 min
Present compound 33: LCMS: 261 [M+H]⁺, RT = 1.21 min
Present compound 34: LCMS: 323 [M+H]⁺, RT = 1.68 min
Present compound 35: LCMS: 357 [M+H]⁺, RT = 1.81 min

Examples of the Present compound prepared according to the above Production methods and Preparation Examples are shown below.

The compound represented by formula (I), wherein n represents 0, J represents J1-1, Q represents Q1-1, and E represents any one substituent described in Table V1 to Table V7 (hereinafter referred to as “Compound group SX1”).

The compound represented by formula (I), wherein n represents 0, J represents J1-2, Q represents Q1-1, and E represents any one substituent described in Table V1 to Table V7 (hereinafter referred to as “Compound group SX2”).

The compound represented by formula (I), wherein n represents 0, J represents J1-3, Q represents Q1-1, and E represents any one substituent described in Table V1 to Table V7 (hereinafter referred to as “Compound group SX3”).

The compound represented by formula (I), wherein n represents 0, J represents J1-4, Q represents Q1-1, and E represents any one substituent described in Table V1 to Table V7 (hereinafter referred to as “Compound group SX4”).

The compound represented by formula (I), wherein n represents 0, J represents J1-5, Q represents Q1-1, and E represents any one substituent described in Table V1 to Table V7 (hereinafter referred to as “Compound group SX5”).

The compound represented by formula (I), wherein n represents 0, J represents J1-6, Q represents Q1-1, and E represents any one substituent described in Table V1 to Table V7 (hereinafter referred to as “Compound group SX6”).

The compound represented by formula (I), wherein n represents 0, J represents J1-7, Q represents Q1-1, and E represents any one substituent described in Table V1 to Table V7 (hereinafter referred to as “Compound group SX7”).

The compound represented by formula (I), wherein n represents 0, J represents J1-8, Q represents Q1-1, and E represents any one substituent described in Table V1 to Table V7 (hereinafter referred to as “Compound group SX8”).

The compound represented by formula (I), wherein n represents 0, J represents J2-1, Q represents Q1-1, and E represents any one substituent described in Table V1 to Table V7 (hereinafter referred to as “Compound group SX9”).

The compound represented by formula (I), wherein n represents 0, J represents J2-2, Q represents Q1-1, and E represents any one substituent described in Table V1 to Table V7 (hereinafter referred to as “Compound group SX10”).

The compound represented by formula (I), wherein n represents 0, J represents J2-3, Q represents Q1-1, and E represents any one substituent described in Table V1 to Table V7 (hereinafter referred to as “Compound group SX11”).

The compound represented by formula (I), wherein n represents 0, J represents J2-4, Q represents Q1-1, and E represents any one substituent described in Table V1 to Table V7 (hereinafter referred to as “Compound group SX12”).

The compound represented by formula (I), wherein n represents 0, J represents J2-5, Q represents Q1-1, and E represents any one substituent described in Table V1 to Table V7 (hereinafter referred to as “Compound group SX13”).

The compound represented by formula (I), wherein n represents 0, J represents J2-6, Q represents Q1-1, and E represents any one substituent described in Table V1 to Table V7 (hereinafter referred to as “Compound group SX14”).

The compound represented by formula (I), wherein n represents 0, J represents J2-7, Q represents Q1-1, and E represents any one substituent described in Table V1 to Table V7 (hereinafter referred to as “Compound group SX15”).

The compound represented by formula (I), wherein n represents 0, J represents J1-1, Q represents Q1-2, and E represents any one substituent described in Table V1 to Table V7 (hereinafter referred to as “Compound group SX16”).

The compound represented by formula (I), wherein n represents 0, J represents J1-2, Q represents Q1-2, and E represents any one substituent described in Table V1 to Table V7 (hereinafter referred to as “Compound group SX17”).

The compound represented by formula (I), wherein n represents 0, J represents J1-3, Q represents Q1-2, and E represents any one substituent described in Table V1 to Table V7 (hereinafter referred to as “Compound group SX18”).

The compound represented by formula (I), wherein n represents 0, J represents J1-4, Q represents Q1-2, and E represents any one substituent described in Table V1 to Table V7 (hereinafter referred to as “Compound group SX19”).

The compound represented by formula (I), wherein n represents 0, J represents J1-5, Q represents Q1-2, and E represents any one substituent described in Table V1 to Table V7 (hereinafter referred to as “Compound group SX20”).

The compound represented by formula (I), wherein n represents 0, J represents J1-6, Q represents Q1-2, and E represents any one substituent described in Table V1 to Table V7 (hereinafter referred to as “Compound group SX21”).

The compound represented by formula (I), wherein n represents 0, J represents J1-7, Q represents Q1-2, and E represents any one substituent described in Table V1 to Table V7 (hereinafter referred to as “Compound group SX22”).

The compound represented by formula (I), wherein n represents 0, J represents J1-8, Q represents Q1-2, and E represents any one substituent described in Table V1 to Table V7 (hereinafter referred to as “Compound group SX23”).

The compound represented by formula (I), wherein n represents 0, J represents J2-1, Q represents Q1-2, and E represents any one substituent described in Table V1 to Table V7 (hereinafter referred to as “Compound group SX24”).

The compound represented by formula (I), wherein n represents 0, J represents J2-2, Q represents Q1-2, and E represents any one substituent described in Table V1 to Table V7 (hereinafter referred to as “Compound group SX25”).

The compound represented by formula (I), wherein n represents 0, J represents J2-3, Q represents Q1-2, and E represents any one substituent described in Table V1 to Table V7 (hereinafter referred to as “Compound group SX26”).

The compound represented by formula (I), wherein n represents 0, J represents J2-4, Q represents Q1-2, and E represents any one substituent described in Table V1 to Table V7 (hereinafter referred to as “Compound group SX27”).

The compound represented by formula (I), wherein n represents 0, J represents J2-5, Q represents Q1-2, and E represents any one substituent described in Table V1 to Table V7 (hereinafter referred to as “Compound group SX28”).

The compound represented by formula (I), wherein n represents 0, J represents J2-6, Q represents Q1-2, and E represents any one substituent described in Table V1 to Table V7 (hereinafter referred to as “Compound group SX29”).

The compound represented by formula (I), wherein n represents 0, J represents J2-7, Q represents Q1-2, and E represents any one substituent described in Table V1 to Table V7 (hereinafter referred to as “Compound group SX30”).

The compound represented by formula (I), wherein n represents 0, J represents J1-1, Q represents Q1-3, and E represents any one substituent described in Table V1 to Table V7 (hereinafter referred to as “Compound group SX31”).

The compound represented by formula (I), wherein n represents 0, J represents J1-2, Q represents Q1-3, and E represents any one substituent described in Table V1 to Table V7 (hereinafter referred to as “Compound group SX32”).

The compound represented by formula (I), wherein n represents 0, J represents J1-3, Q represents Q1-3, and E represents any one substituent described in Table V1 to Table V7 (hereinafter referred to as “Compound group SX33”).

The compound represented by formula (I), wherein n represents 0, J represents J1-4, Q represents Q1-3, and E represents any one substituent described in Table V1 to Table V7 (hereinafter referred to as “Compound group SX34”).

The compound represented by formula (I), wherein n represents 0, J represents J1-5, Q represents Q1-3, and E represents any one substituent described in Table V1 to Table V7 (hereinafter referred to as “Compound group SX35”).

The compound represented by formula (I), wherein n represents 0, J represents J1-6, Q represents Q1-3, and E represents any one substituent described in Table V1 to Table V7 (hereinafter referred to as “Compound group SX36”).

The compound represented by formula (I), wherein n represents 0, J represents J1-7, Q represents Q1-3, and E represents any one substituent described in Table V1 to Table V7 (hereinafter referred to as “Compound group SX37”).

The compound represented by formula (I), wherein n represents 0, J represents J1-8, Q represents Q1-3, and E represents any one substituent described in Table V1 to Table V7 (hereinafter referred to as “Compound group SX38”).

The compound represented by formula (I), wherein n represents 0, J represents J2-1, Q represents Q1-3, and E represents any one substituent described in Table V1 to Table V7 (hereinafter referred to as “Compound group SX39”).

The compound represented by formula (I), wherein n represents 0, J represents J2-2, Q represents Q1-3, and E represents any one substituent described in Table V1 to Table V7 (hereinafter referred to as “Compound group SX40”).

The compound represented by formula (I), wherein n represents 0, J represents J2-3, Q represents Q1-3, and E represents any one substituent described in Table V1 to Table V7 (hereinafter referred to as “Compound group SX41”).

The compound represented by formula (I), wherein n represents 0, J represents J2-4, Q represents Q1-3, and E represents any one substituent described in Table V1 to Table V7 (hereinafter referred to as “Compound group SX42”).

The compound represented by formula (I), wherein n represents 0, J represents J2-5, Q represents Q1-3, and E represents any one substituent described in Table V1 to Table V7 (hereinafter referred to as “Compound group SX43”).

The compound represented by formula (I), wherein n represents 0, J represents J2-6, Q represents Q1-3, and E represents any one substituent described in Table V1 to Table V7 (hereinafter referred to as “Compound group SxX44”).

The compound represented by formula (I), wherein n represents 0, J represents J2-7, Q represents Q1-3, and E represents any one substituent described in Table V1 to Table V7 (hereinafter referred to as “Compound group SX45”).

The compound represented by formula (I), wherein n represents 0, J represents J1-1, Q represents Q1-4, and E represents any one substituent described in Table V1 to Table V7 (hereinafter referred to as “Compound group SX46”).

The compound represented by formula (I), wherein n represents 0, J represents J1-2, Q represents Q1-4, and E represents any one substituent described in Table V1 to Table V7 (hereinafter referred to as “Compound group SX47”).

The compound represented by formula (I), wherein n represents 0, J represents J1-3, Q represents Q1-4, and E represents any one substituent described in Table V1 to Table V7 (hereinafter referred to as “Compound group SX48”).

The compound represented by formula (I), wherein n represents 0, J represents J1-4, Q represents Q1-4, and E represents any one substituent described in Table V1 to Table V7 (hereinafter referred to as “Compound group SX49”).

The compound represented by formula (I), wherein n represents 0, J represents J1-5, Q represents Q1-4, and E represents any one substituent described in Table V1 to Table V7 (hereinafter referred to as “Compound group SX50”).

The compound represented by formula (I), wherein n represents 0, J represents J1-6, Q represents Q1-4, and E represents any one substituent described in Table V1 to Table V7 (hereinafter referred to as “Compound group SX51”).

The compound represented by formula (I), wherein n represents 0, J represents J1-7, Q represents Q1-4, and E represents any one substituent described in Table V1 to Table V7 (hereinafter referred to as “Compound group SX52”).

The compound represented by formula (I), wherein n represents 0, J represents J1-8, Q represents Q1-4, and E represents any one substituent described in Table V1 to Table V7 (hereinafter referred to as “Compound group SX53”).

The compound represented by formula (I), wherein n represents 0, J represents J2-1, Q represents Q1-4, and E represents any one substituent described in Table V1 to Table V7 (hereinafter referred to as “Compound group SX54”).

The compound represented by formula (I), wherein n represents 0, J represents J2-2, Q represents Q1-4, and E represents any one substituent described in Table V1 to Table V7 (hereinafter referred to as “Compound group SX55”).

The compound represented by formula (I), wherein n represents 0, J represents J2-3, Q represents Q1-4, and E represents any one substituent described in Table V1 to Table V7 (hereinafter referred to as “Compound group SX56”).

The compound represented by formula (I), wherein n represents 0, J represents J2-4, Q represents Q1-4, and E represents any one substituent described in Table V1 to Table V7 (hereinafter referred to as “Compound group SX57”).

The compound represented by formula (I), wherein n represents 0, J represents J2-5, Q represents Q1-4, and E represents any one substituent described in Table V1 to Table V7 (hereinafter referred to as “Compound group SX58”).

The compound represented by formula (I), wherein n represents 0, J represents J2-6, Q represents Q1-4, and E represents any one substituent described in Table V1 to Table V7 (hereinafter referred to as “Compound group SX59”).

The compound represented by formula (I), wherein n represents 0, J represents J2-7, Q represents Q1-4, and E represents any one substituent described in Table V1 to Table V7 (hereinafter referred to as “Compound group SX60”).

The compound represented by formula (I), wherein n represents 0, J represents J1-1, Q represents Q1-5, and E represents any one substituent described in Table V1 to Table V7 (hereinafter referred to as “Compound group SX61”).

The compound represented by formula (I), wherein n represents 0, J represents J1-2, Q represents Q1-5, and E represents any one substituent described in Table V1 to Table V7 (hereinafter referred to as “Compound group SX62”).

The compound represented by formula (I), wherein n represents 0, J represents J1-3, Q represents Q1-5, and E represents any one substituent described in Table V1 to Table V7 (hereinafter referred to as “Compound group SX63”).

The compound represented by formula (I), wherein n represents 0, J represents J1-4, Q represents Q1-5, and E represents any one substituent described in Table V1 to Table V7 (hereinafter referred to as “Compound group SX64”).

The compound represented by formula (I), wherein n represents 0, J represents J1-5, Q represents Q1-5, and E represents any one substituent described in Table V1 to Table V7 (hereinafter referred to as “Compound group SX65”).

The compound represented by formula (I), wherein n represents 0, J represents J1-6, Q represents Q1-5, and E represents any one substituent described in Table V1 to Table V7 (hereinafter referred to as “Compound group SX66”).

The compound represented by formula (I), wherein n represents 0, J represents J1-7, Q represents Q1-5, and E represents any one substituent described in Table V1 to Table V7 (hereinafter referred to as “Compound group SX67”).

The compound represented by formula (I), wherein n represents 0, J represents J1-8, Q represents Q1-5, and E represents any one substituent described in Table V1 to Table V7 (hereinafter referred to as “Compound group SX68”).

The compound represented by formula (I), wherein n represents 0, J represents J2-1, Q represents Q1-5, and E represents any one substituent described in Table V1 to Table V7 (hereinafter referred to as “Compound group SX69”).

The compound represented by formula (I), wherein n represents 0, J represents J2-2, Q represents Q1-5, and E represents any one substituent described in Table V1 to Table V7 (hereinafter referred to as “Compound group SX70”).

The compound represented by formula (I), wherein n represents 0, J represents J2-3, Q represents Q1-5, and E represents any one substituent described in Table V1 to Table V7 (hereinafter referred to as “Compound group SX71”).

The compound represented by formula (I), wherein n represents 0, J represents J2-4, Q represents Q1-5, and E represents any one substituent described in Table V1 to Table V7 (hereinafter referred to as “Compound group SX72”).

The compound represented by formula (I), wherein n represents 0, J represents J2-5, Q represents Q1-5, and E represents any one substituent described in Table V1 to Table V7 (hereinafter referred to as “Compound group SX73”).

The compound represented by formula (I), wherein n represents 0, J represents J2-6, Q represents Q1-5, and E represents any one substituent described in Table V1 to Table V7 (hereinafter referred to as “Compound group SX74”).

The compound represented by formula (I), wherein n represents 0, J represents J2-7, Q represents Q1-5, and E represents any one substituent described in Table V1 to Table V7 (hereinafter referred to as “Compound group SX75”).

The compound represented by formula (I), wherein n represents 0, J represents J1-1, Q represents Q1-6, and E represents any one substituent described in Table V1 to Table V7 (hereinafter referred to as “Compound group SX76”).

The compound represented by formula (I), wherein n represents 0, J represents J1-2, Q represents Q1-6, and E represents any one substituent described in Table V1 to Table V7 (hereinafter referred to as “Compound group SX77”).

The compound represented by formula (I), wherein n represents 0, J represents J1-3, Q represents Q1-6, and E represents any one substituent described in Table V1 to Table V7 (hereinafter referred to as “Compound group SX78”).

The compound represented by formula (I), wherein n represents 0, J represents J1-4, Q represents Q1-6, and E represents any one substituent described in Table V1 to Table V7 (hereinafter referred to as “Compound group SX79”).

The compound represented by formula (I), wherein n represents 0, J represents J1-5, Q represents Q1-6, and E represents any one substituent described in Table V1 to Table V7 (hereinafter referred to as “Compound group SX80”).

The compound represented by formula (I), wherein n represents 0, J represents J1-6, Q represents Q1-6, and E represents any one substituent described in Table V1 to Table V7 (hereinafter referred to as “Compound group SX81”).

The compound represented by formula (I), wherein n represents 0, J represents J1-7, Q represents Q1-6, and E represents any one substituent described in Table V1 to Table V7 (hereinafter referred to as “Compound group SX82”).

The compound represented by formula (I), wherein n represents 0, J represents J1-8, Q represents Q1-6, and E represents any one substituent described in Table V1 to Table V7 (hereinafter referred to as “Compound group SX83”).

The compound represented by formula (I), wherein n represents 0, J represents J2-1, Q represents Q1-6, and E represents any one substituent described in Table V1 to Table V7 (hereinafter referred to as “Compound group SX84”).

The compound represented by formula (I), wherein n represents 0, J represents J2-2, Q represents Q1-6, and E represents any one substituent described in Table V1 to Table V7 (hereinafter referred to as “Compound group SX85”).

The compound represented by formula (I), wherein n represents 0, J represents J2-3, Q represents Q1-6, and E represents any one substituent described in Table V1 to Table V7 (hereinafter referred to as “Compound group SX86”).

The compound represented by formula (I), wherein n represents 0, J represents J2-4, Q represents Q1-6, and E represents any one substituent described in Table V1 to Table V7 (hereinafter referred to as “Compound group SX87”).

The compound represented by formula (I), wherein n represents 0, J represents J2-5, Q represents Q1-6, and E represents any one substituent described in Table V1 to Table V7 (hereinafter referred to as “Compound group SX88”).

The compound represented by formula (I), wherein n represents 0, J represents J2-6, Q represents Q1-6, and E represents any one substituent described in Table V1 to Table V7 (hereinafter referred to as “Compound group SX89”).

The compound represented by formula (I), wherein n represents 0, J represents J2-7, Q represents Q1-6, and E represents any one substituent described in Table V1 to Table V7 (hereinafter referred to as “Compound group SX90”).

The compound represented by formula (I), wherein n represents 0, J represents J1-1, Q represents Q1-7, and E represents any one substituent described in Table V1 to Table V7 (hereinafter referred to as “Compound group SX91”).

The compound represented by formula (I), wherein n represents 0, J represents J1-2, Q represents Q1-7, and E represents any one substituent described in Table V1 to Table V7 (hereinafter referred to as “Compound group SX92”).

The compound represented by formula (I), wherein n represents 0, J represents J1-3, Q represents Q1-7, and E represents any one substituent described in Table V1 to Table V7 (hereinafter referred to as “Compound group SX93”).

The compound represented by formula (I), wherein n represents 0, J represents J1-4, Q represents Q1-7, and E represents any one substituent described in Table V1 to Table V7 (hereinafter referred to as “Compound group SX94”).

The compound represented by formula (I), wherein n represents 0, J represents J1-5, Q represents Q1-7, and E represents any one substituent described in Table V1 to Table V7 (hereinafter referred to as “Compound group SX95”).

The compound represented by formula (I), wherein n represents 0, J represents J1-6, Q represents Q1-7, and E represents any one substituent described in Table V1 to Table V7 (hereinafter referred to as “Compound group SX96”).

The compound represented by formula (I), wherein n represents 0, J represents J1-7, Q represents Q1-7, and E represents any one substituent described in Table V1 to Table V7 (hereinafter referred to as “Compound group SX97”).

The compound represented by formula (I), wherein n represents 0, J represents J1-8, Q represents Q1-7, and E represents any one substituent described in Table V1 to Table V7 (hereinafter referred to as “Compound group SX98”).

The compound represented by formula (I), wherein n represents 0, J represents J2-1, Q represents Q1-7, and E represents any one substituent described in Table V1 to Table V7 (hereinafter referred to as “Compound group SX99”).

The compound represented by formula (I), wherein n represents 0, J represents J2-2, Q represents Q1-7, and E represents any one substituent described in Table V1 to Table V7 (hereinafter referred to as “Compound group SX100”).

The compound represented by formula (I), wherein n represents 0, J represents J2-3, Q represents Q1-7, and E represents any one substituent described in Table V1 to Table V7 (hereinafter referred to as “Compound group SX101”).

The compound represented by formula (I), wherein n represents 0, J represents J2-4, Q represents Q1-7, and E represents any one substituent described in Table V1 to Table V7 (hereinafter referred to as “Compound group SX102”).

The compound represented by formula (I), wherein n represents 0, J represents J2-5, Q represents Q1-7, and E represents any one substituent described in Table V1 to Table V7 (hereinafter referred to as “Compound group SX103”).

The compound represented by formula (I), wherein n represents 0, J represents J2-6, Q represents Q1-7, and E represents any one substituent described in Table V1 to Table V7 (hereinafter referred to as “Compound group SX104”).

The compound represented by formula (I), wherein n represents 0, J represents J2-7, Q represents Q1-7, and E represents any one substituent described in Table V1 to Table V7 (hereinafter referred to as “Compound group SX105”).

The compound represented by formula (I), wherein n represents 0, J represents J1-1, Q represents Q1-8, and E represents any one substituent described in Table V1 to Table V7 (hereinafter referred to as “Compound group SX106”).

The compound represented by formula (I), wherein n represents 0, J represents J1-2, Q represents Q1-8, and E represents any one substituent described in Table V1 to Table V7 (hereinafter referred to as “Compound group SX107”) .

The compound represented by formula (I), wherein n represents 0, J represents J1-3, Q represents Q1-8, and E represents any one substituent described in Table V1 to Table V7 (hereinafter referred to as “Compound group SX108”).

The compound represented by formula (I), wherein n represents 0, J represents J1-4, Q represents Q1-8, and E represents any one substituent described in Table V1 to Table V7 (hereinafter referred to as “Compound group SX109”).

The compound represented by formula (I), wherein n represents 0, J represents J1-5, Q represents Q1-8, and E represents any one substituent described in Table V1 to Table V7 (hereinafter referred to as “Compound group SX110”).

The compound represented by formula (I), wherein n represents 0, J represents J1-6, Q represents Q1-8, and E represents any one substituent described in Table V1 to Table V7 (hereinafter referred to as “Compound group SX111”).

The compound represented by formula (I), wherein n represents 0, J represents J1-7, Q represents Q1-8, and E represents any one substituent described in Table V1 to Table V7 (hereinafter referred to as “Compound group SX112”) .

The compound represented by formula (I), wherein n represents 0, J represents J1-8, Q represents Q1-8, and E represents any one substituent described in Table V1 to Table V7 (hereinafter referred to as “Compound group SX113”).

The compound represented by formula (I), wherein n represents 0, J represents J2-1, Q represents Q1-8, and E represents any one substituent described in Table V1 to Table V7 (hereinafter referred to as “Compound group SX114”).

The compound represented by formula (I), wherein n represents 0, J represents J2-2, Q represents Q1-8, and E represents any one substituent described in Table V1 to Table V7 (hereinafter referred to as “Compound group SX115”).

The compound represented by formula (I), wherein n represents 0, J represents J2-3, Q represents Q1-8, and E represents any one substituent described in Table V1 to Table V7 (hereinafter referred to as “Compound group SX116”).

The compound represented by formula (I), wherein n represents 0, J represents J2-4, Q represents Q1-8, and E represents any one substituent described in Table V1 to Table V7 (hereinafter referred to as “Compound group SX117”).

The compound represented by formula (I), wherein n represents 0, J represents J2-5, Q represents Q1-8, and E represents any one substituent described in Table V1 to Table V7 (hereinafter referred to as “Compound group SX118”).

The compound represented by formula (I), wherein n represents 0, J represents J2-6, Q represents Q1-8, and E represents any one substituent described in Table V1 to Table V7 (hereinafter referred to as “Compound group SX119”).

The compound represented by formula (I), wherein n represents 0, J represents J2-7, Q represents Q1-8, and E represents any one substituent described in Table V1 to Table V7 (hereinafter referred to as “Compound group SX120”).

The compound represented by formula (I), wherein n represents 0, J represents J1-1, Q represents Q2-1, and E represents any one substituent described in Table V1 to Table V7 (hereinafter referred to as “Compound group SX121”).

The compound represented by formula (I), wherein n represents 0, J represents J1-2, Q represents Q2-1, and E represents any one substituent described in Table V1 to Table V7 (hereinafter referred to as “Compound group SX122”).

The compound represented by formula (I), wherein n represents 0, J represents J1-3, Q represents Q2-1, and E represents any one substituent described in Table V1 to Table V7 (hereinafter referred to as “Compound group SX123”).

The compound represented by formula (I), wherein n represents 0, J represents J1-4, Q represents Q2-1, and E represents any one substituent described in Table V1 to Table V7 (hereinafter referred to as “Compound group SX124”).

The compound represented by formula (I), wherein n represents 0, J represents J1-5, Q represents Q2-1, and E represents any one substituent described in Table V1 to Table V7 (hereinafter referred to as “Compound group SX125”).

The compound represented by formula (I), wherein n represents 0, J represents J1-6, Q represents Q2-1, and E represents any one substituent described in Table V1 to Table V7 (hereinafter referred to as “Compound group SX126”).

The compound represented by formula (I), wherein n represents 0, J represents J1-7, Q represents Q2-1, and E represents any one substituent described in Table V1 to Table V7 (hereinafter referred to as “Compound group SX127”).

The compound represented by formula (I), wherein n represents 0, J represents J1-8, Q represents Q2-1, and E represents any one substituent described in Table V1 to Table V7 (hereinafter referred to as “Compound group SX128”).

The compound represented by formula (I), wherein n represents 0, J represents J2-1, Q represents Q2-1, and E represents any one substituent described in Table V1 to Table V7 (hereinafter referred to as “Compound group SX129”).

The compound represented by formula (I), wherein n represents 0, J represents J2-2, Q represents Q2-1, and E represents any one substituent described in Table V1 to Table V7 (hereinafter referred to as “Compound group SX130”).

The compound represented by formula (I), wherein n represents 0, J represents J2-3, Q represents Q2-1, and E represents any one substituent described in Table V1 to Table V7 (hereinafter referred to as “Compound group SX131”).

The compound represented by formula (I), wherein n represents 0, J represents J2-4, Q represents Q2-1, and E represents any one substituent described in Table V1 to Table V7 (hereinafter referred to as “Compound group SX132”).

The compound represented by formula (I), wherein n represents 0, J represents J2-5, Q represents Q2-1, and E represents any one substituent described in Table V1 to Table V7 (hereinafter referred to as “Compound group SX133”).

The compound represented by formula (I), wherein n represents 0, J represents J2-6, Q represents Q2-1, and E represents any one substituent described in Table V1 to Table V7 (hereinafter referred to as “Compound group SX134”).

The compound represented by formula (I), wherein n represents 0, J represents J2-7, Q represents Q2-1, and E represents any one substituent described in Table V1 to Table V7 (hereinafter referred to as “Compound group SX135”).

The compound represented by formula (I), wherein n represents 0, J represents J1-1, Q represents Q2-2, and E represents any one substituent described in Table V1 to Table V7 (hereinafter referred to as “Compound group SX136”).

The compound represented by formula (I), wherein n represents 0, J represents J1-2, Q represents Q2-2, and E represents any one substituent described in Table V1 to Table V7 (hereinafter referred to as “Compound group SX137”).

The compound represented by formula (I), wherein n represents 0, J represents J1-3, Q represents Q2-2, and E represents any one substituent described in Table V1 to Table V7 (hereinafter referred to as “Compound group SX138”).

The compound represented by formula (I), wherein n represents 0, J represents J1-4, Q represents Q2-2, and E represents any one substituent described in Table V1 to Table V7 (hereinafter referred to as “Compound group SX139”).

The compound represented by formula (I), wherein n represents 0, J represents J1-5, Q represents Q2-2, and E represents any one substituent described in Table V1 to Table V7 (hereinafter referred to as “Compound group SX140”).

The compound represented by formula (I), wherein n represents 0, J represents J1-6, Q represents Q2-2, and E represents any one substituent described in Table V1 to Table V7 (hereinafter referred to as “Compound group SX141”).

The compound represented by formula (I), wherein n represents 0, J represents J1-7, Q represents Q2-2, and E represents any one substituent described in Table V1 to Table V7 (hereinafter referred to as “Compound group SX142”).

The compound represented by formula (I), wherein n represents 0, J represents J1-8, Q represents Q2-2, and E represents any one substituent described in Table V1 to Table V7 (hereinafter referred to as “Compound group SX143”).

The compound represented by formula (I), wherein n represents 0, J represents J2-1, Q represents Q2-2, and E represents any one substituent described in Table V1 to Table V7 (hereinafter referred to as “Compound group SX144”).

The compound represented by formula (I), wherein n represents 0, J represents J2-2, Q represents Q2-2, and E represents any one substituent described in Table V1 to Table V7 (hereinafter referred to as “Compound group SX145”).

The compound represented by formula (I), wherein n represents 0, J represents J2-3, Q represents Q2-2, and E represents any one substituent described in Table V1 to Table V7 (hereinafter referred to as “Compound group SX146”).

The compound represented by formula (I), wherein n represents 0, J represents J2-4, Q represents Q2-2, and E represents any one substituent described in Table V1 to Table V7 (hereinafter referred to as “Compound group SX147”).

The compound represented by formula (I), wherein n represents 0, J represents J2-5, Q represents Q2-2, and E represents any one substituent described in Table V1 to Table V7 (hereinafter referred to as “Compound group SX148”).

The compound represented by formula (I), wherein n represents 0, J represents J2-6, Q represents Q2-2, and E represents any one substituent described in Table V1 to Table V7 (hereinafter referred to as “Compound group SX149”).

The compound represented by formula (I), wherein n represents 0, J represents J2-7, Q represents Q2-2, and E represents any one substituent described in Table V1 to Table V7 (hereinafter referred to as “Compound group SX150”).

Next, Formulation Examples are shown below. The “part(s)” in Formulation Examples represents “part(s) by weight”. Also, the expression of “Present compound S” represents the compounds described in the Compound groups SX1 to SX150.

### Formulation Example 1

A mixture of polyoxyethylene alkyl ether sulfate ammonium salt and silica (weight ratio of 1 : 1) (35 parts), any one of the Present compound S (10 parts), and water (55 parts) are mixed, and the resulting mixture is subjected to fine grinding according to a wet grinding method to obtain each formulation.

### Formulation Example 2

Any one of the Present compound S (50 parts), calcium lignin sulfonate (3 parts), sodium lauryl sulfate (2 parts), and silica (45 parts) are ground and mixed to obtain each formulation.

### Formulation Example 3

Any one of the Present compound S (5 parts), polyoxyethylene styryl phenyl ether (9 parts), polyoxyethylene decyl ether (number of added ethyleneoxide: 5) (5 parts), calcium dodecylbenzene sulfonate (6 parts), and xylene (75 parts) are mixed to obtain each formulation.

### Formulation Example 4

Any one of the Present compound S (2 parts), silica (1 part), calcium lignin sulfonate (2 parts), bentonite (30 parts), and kaolin clay (65 parts) are ground and mixed, an appropriate amount of water is added thereto, the resulting mixture is kneaded, subjected to granulation with a granulator, and then dried to obtain each formulation.

Next, Test Examples are shown below.

### Test Example 1

A true leaf of a soybean (cultivar: Kurosengoku) was cut into a diameter of 1 cm to prepare each leaf disc. To each well of a 24 well microplate was dispensed 1 mL of an agar medium (agar concentration 1.2%), and then one of said leaf disc was placed on each well. To a mixture of Sorpol (registered trademark) 1200KX (0.5 µL), DMSO (4.5 µL), and xylene (5 µL) was added a DMSO solution (20 µL) comprising a test compound (10000 ppm), and the resulting mixture was mixed. The resulting mixture was diluted with ion exchange water to prepare a spray solution comprising a prescribed concentration of the test compound. Said spray solution was sprayed into each leaf disc at a ratio of 10 µL per leaf disc. After 1 day, an aqueous suspension of spores of soybean rust fungus (Phakopsora pachyrhizi) having an amino acid substitution of F129L in a mitochondrial cytochrome b protein (1.0 × 10⁵/mL) was inoculated by spraying on each leaf disc. After the inoculation, the microplate was placed into an artificial climate chamber (Lighting: 6 hours, Lights-out: 18 hours, Temperature: 23° C., Humidity: 60%). After 1 day, the leaf discs were air-dried until water droplets on the surfaces of the leaf discs disappeared, and the microplate was placed into the artificial climate chamber again for 12 days. Then, lesion area of soybean rust was investigated. As a result, when the prescribed concentration was 50 ppm, each lesion area of leaf disc treated with any one of the Present compound 2, 6, 8, 25, 29, or 35 as the test compound was 30% or less relative to the lesion area of non-treated leaf disc.

Here, the term of “non-treated” means that a spray solution comprising a test compound was not sprayed into a leaf disc.

### Test Example 2

Each test is carried out according to the Test Example 1 by using any one of the Present compound S as the test compound with a prescribed concentration of 12.5 ppm. As a result, control effects on soybean rust can be confirmed in the leaf discs treated with test compounds.

### Test Example 3

Each test is carried out according to the Test Example 1 by using any one of the Present compound S as the test compound with a prescribed concentration of 3.1 ppm. As a result, control effects on soybean rust can be confirmed in the leaf discs treated with test compounds.

### Comparative Test Example 1

Each test was carried out according to the Test Example 1 by using the Present compound 2, 6, 8, 29 or 35, or azoxystrobin, dimoxystrobin, or metominostrobin as the test compound with a prescribed concentration of 50 ppm. The results are shown in Table A.

TABLE A

| | |
|---|---|
| Compound | Lesion area (%) at the concentration of 50 ppm |
| 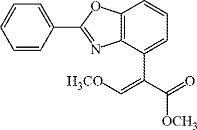 [SMILES: CO/C=C(/C(=O)OC)c1cccc2oc(-c3ccccc3)nc12] | 10 |
| Present compound 2 | |
| 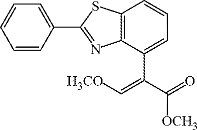 [SMILES: CO/C=C(/C(=O)OC)c1cccc2sc(-c3ccccc3)nc12] | 30 |
| Present compound 6 | |
| 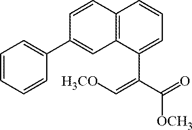 [SMILES: CO/C=C(/C(=O)OC)c1cccc2ccc(-c3ccccc3)cc12] | 0 |
| Present compound 8 | |
| 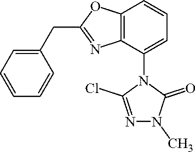 [SMILES: Cn1nc(Cl)n(-c2cccc3oc(Cc4ccccc4)nc23)c1=O] | 10 |
| Present compound 29 | |
| 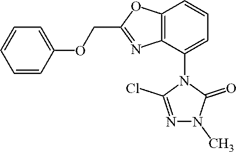 [SMILES: Cn1nc(Cl)n(-c2cccc3oc(COc4ccccc4)nc23)c1=O] | 0 |
| Present compound 35 | |
| 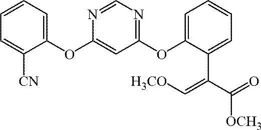 [SMILES: CO/C=C(/C(=O)OC)c1ccccc1Oc1cc(Oc2ccccc2C#N)ncn1] | 100 |
| Azoxystrobin | |
| 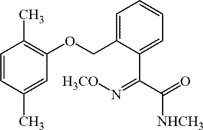 [SMILES: CNC(=O)/C(=N/OC)c1ccccc1COc1cc(C)ccc1C] | 100 |
| Dimoxystrobin | |
| 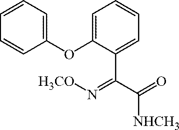 [SMILES: CNC(=O)/C(=N/OC)c1ccccc1Oc1ccccc1] | 100 |
| Metominostrobin | |

The above results demonstrate that the Present compound has excellent activities against soybean rust fungi having an amino acid substitution of F129L as compared to several commercially available QoI fungicides.

### INDUSTRIAL APPLICABILITY

The Present compo und can be used for controlling soybean rust fungi having an amino acid substitution of F129L in a mitochondrial cytochrome b protein.

1. A method for controlling a soybean rust fungus having an amino acid substitution of F129L in a mitochondrial cytochrome b protein, which comprises applying an effective amount of a compound represented by formula (I) 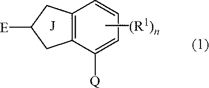
[wherein:
R¹ represents a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, or a hydroxy group;
n represents 0, 1, or 2;
when n represents 2, two R¹ may be identical to or different from each other;
Q represents a group represented by Q1 or a group represented by Q2; 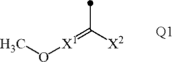 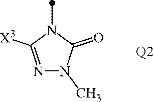
• represents the binding site to the rest of molecule;
X¹ represents —C(H)═ or —N═;
X² represents —C(O)OCH₃, —C(O)NHCH₃, or a 5,6-dihydro-1,4,2-dioxazin-3-yl group;
X³ represents a C1-C3 chain hydrocarbon group, a cyclopropyl group, a C1-C3 alkoxy group {wherein said C1-C3 chain hydrocarbon group, said cyclopropyl group, and said C1-C3 alkoxy group are optionally substituted with one or more halogen atom(s)}, or a halogen atom;
J represents a group represented by J1 or a group represented by J2; 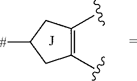 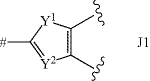 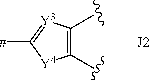
# represents the binding position to E;
Y¹ represents an oxygen atom, a sulfur atom, —N(R²)—, *—C(R³)═C(R⁴)—, or *-N=C(R⁵_{)-;}
* represents the binding position to the carbon atom bound to E;
Y² represents ═C(R⁶)— or ═N—;
Y³ represents ═C(R⁷)— or ═N—;
Y⁴ represents an oxygen atom, a sulfur atom, or —N(R⁸)—;
R² and R⁸ are identical to or different from each other, and each represent a C1-C3 chain hydrocarbon group, a cyclopropyl group {wherein said C1-C3 chain hydrocarbon group and said cyclopropyl group are optionally substituted with one or more halogen atom(s)}, or a hydrogen atom;
R³, R⁴, R⁵, R⁶, and R⁷ are identical to or different from each other, and each represent a C1-C4 alkyl group, a C1-C4 alkoxy group {wherein said C1-C4 alkyl group and said C1-C4 alkoxy group are optionally substituted with one or more halogen atom(s)}, a cyano group, a nitro group, a halogen atom, a hydroxy group, or a hydrogen atom;
E represents a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group D}, R⁹—L¹—CH₂—, R¹⁰R¹¹C═N—O—CH₂—, R¹²O—N═C(R¹³)—C(R¹⁴)═N—O—CH₂—, R¹⁵C(O)—C(R¹⁶)═N—O—CH₂—, R¹⁷R¹⁸N—C(S)—O—CH₂₋, R¹⁹N═C(R²⁰)—S—CH₂—, R²¹N═C(SR²²)—S—CH₂—, R²³O—N═C(R²⁴)—S—CH₂—, R²⁵O—N═C(SR²⁶)—S—CH₂—, R²⁷O—N═C(R²⁸)—, R²⁹R³⁰C═N—N═C(R³¹)—, R³²R³³N—N═C(R³⁴)—, R³⁵—N═C(R³⁶)—, R³⁷SC(R³⁸)═N—, R³⁹SC(SR⁴⁰)═N—, R⁴¹L²-, R⁴³C(O)O—, R⁴⁴OC(O)O—, R⁴⁵R⁴⁶NC(O)O-, R⁴⁷R⁴⁸NC(S)O-, R⁴⁹S(O)₂O—, R⁵⁰R⁵¹NS(O)₂O-, a cyano group, a nitro group, a hydroxy group, or a halogen atom;
L¹ and L² are identical to or different from each other, and each represent an oxygen atom or a sulfur atom;
R⁹ represents a C6-C10 aryl group or a 5-10 membered aromatic heterocyclic group {wherein said C6-C10 aryl group and said 5-10 membered aromatic heterocyclic group are optionally substituted with one or more substituent(s) selected from Group D};
R¹², R¹⁵, R¹⁷, R¹⁹, R²¹, R²³, R²⁵, R²⁹, R³², R³⁷, R³⁹, R⁴³, R⁴⁴, R⁴⁵, R⁴⁷, R⁴⁹, and > R⁵⁰ are identical to or different from each other, and each represent a C1-C6 chain hydrocarbon group optionally substituted with one or more substituent(s) selected from Group F, a C3-C10 alicyclic hydrocarbon group optionally substituted with one or more substituent(s) selected from Group B, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group C};
R¹⁰, R²⁷, R³⁵, and R⁴¹ are identical to or different from each other, and each represent a C1-C6 chain hydrocarbon group optionally substituted with one or more substituent(s) selected from Group A, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, or a 5-10 membered aromatic heterocyclic group {wherein said C6-C10 aryl group and said 5-10 membered aromatic heterocyclic group are optionally substituted with one or more substituent(s) selected from Group D};
R¹¹_{,} R¹³, R¹⁴, R¹⁶, R¹⁸, R²⁰, R²², R²⁴, R²⁶, R²⁸, R³⁰, R³¹, R³³, R³⁴, R³⁶, R³⁸, R⁴⁰, R⁴⁶, R⁴⁸, and R⁵¹ are identical to or different from each other, and each represent a C1-C3 chain hydrocarbon group optionally substituted with one or more halogen atom(s), a cyclopropyl group, or a hydrogen atom;
R¹⁰ and R¹¹ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}; and
R²⁹ and R³⁰ are optionally combined with the carbon atom to which they are attached to form a C3-C10 alicyclic hydrocarbon group or a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B};
Group A: a group consisting of a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C1-C4 alkoxy group, a C1-C4 alkylthio group {wherein said C1-C4 alkoxy group and said C1-C4 alkylthio group are optionally substituted with one or more substituent(s) selected from Group F}, a halogen atom, a cyano group, a nitro group, a hydroxy group, an oxo group, a thioxo group, a C6-C10 aryl group, and a 5-10 membered aromatic heterocyclic group {wherein said C6-C10 aryl group and said 5-10 membered aromatic heterocyclic group are optionally substituted with one or more substituent(s) selected from Group D};
Group B: a group consisting of an oxo group, a thioxo group, a C1-C3 chain hydrocarbon group, a C1-C3 alkoxy group {wherein said C1-C3 chain hydrocarbon group and said C1-C3 alkoxy group are optionally substituted with one or more halogen atom(s)}, a halogen atom, and a cyano group;
Group C: a group consisting of a C1-C6 chain hydrocarbon group, a C1-C6 alkoxy group, a C1-C6 alkylthio group {wherein said C1-C6 chain hydrocarbon group, said C1-C6 alkoxy group, and said C1-C6 alkylthio group are optionally substituted with one or more substituent(s) selected from Group F}, a C3-C6 cycloalkyl group {wherein said C3-C6 cycloalkyl group is optionally substituted with one or more substituent(s) selected from Group B}, a cyano group, a nitro group, a halogen atom, and a hydroxy group;
Group D: a group consisting of a C1-C6 chain hydrocarbon group, a C1-C6 alkoxy group, a C1-C6 alkylthio group, a C1-C6 alkylamino group, a C2-C8 dialkylamino group, a (C1-C6 alkyl)carbonyl group, a (C1-C6 alkoxy)carbonyl group, a (C1-C6 alkylamino)carbonyl group, a (C2-C8 dialkylamino)carbonyl group {wherein said C1-C6 chain hydrocarbon group, said C1-C6 alkoxy group, said C1-C6 alkylthio group, said C1-C6 alkylamino group, said C2-C8 dialkylamino group, said (C1-C6 alkyl)carbonyl group, said (C1-C6 alkoxy)carbonyl group, said (C1-C6 alkylamino)carbonyl group, and said (C2-C8 dialkylamino)carbonyl group are optionally substituted with one or more substituent(s) selected from Group F}, a C3-C10 alicyclic hydrocarbon group, a 3-10 membered nonaromatic heterocyclic group {wherein said C3-C10 alicyclic hydrocarbon group and said 3-10 membered nonaromatic heterocyclic group are optionally substituted with one or more substituent(s) selected from Group B}, a C6-C10 aryl group, a 5-10 membered aromatic heterocyclic group {wherein said C6-C10 aryl group and said 5-10 membered aromatic heterocyclic group are optionally substituted with one or more substituent(s) selected from Group C}, a halogen atom, a cyano group, a nitro group, a hydroxy group, and an amino group;
Group F: a group consisting of a C3-C4 cycloalkyl group, a halogen atom, and a C1-C3 alkoxy group]
or an N-oxide or an agriculturally acceptable salt thereof to a soybean or soil for cultivating a soybean.

2. The method according to claim 1, wherein
Q represents the group represented by Q1;
J represents the group represented by J1; and
n represents 0
in the compound represented by formula (1), or an N-oxide or an agriculturally acceptable salt thereof.

3. The method according to claim 1, wherein
E represents a C1-C6 alkyl group optionally substituted with one or more substituent(s) selected from Group A, a C3-C6 cycloalkyl group {wherein said C3-C6 cycloalkyl group is optionally substituted with one or more substituent(s) selected from the group consisting of a C1-C3 alkyl group and a halogen atom}, a phenyl group, a 5-6 membered aromatic heterocyclic group {wherein said phenyl group and said 5-6 membered aromatic heterocyclic group are optionally substituted with one or more substituent(s) selected from Group D}, R⁹—L¹—CH₂—,R⁴¹L²-, or a halogen atom
in the compound represented by formula (I), or an N-oxide or an agriculturally acceptable salt thereof.

4. (canceled)
